(19) United States
(12) Patent Application Publication
(10) Pub. No.: US 2020/0043035 A1
(43) Pub. Date: Feb. 6, 2020

(54) SYSTEM FOR DATA PROCESSING OF HEALTHCARE SERVICE REQUESTS AND DIAGNOSTIC CODES

(71) Applicant: FLIPT, LLC, Jersey City, NJ (US)

(72) Inventors: Lev Peysekhman, Freehold, NJ (US); Bradley Alan Gambill, Haverford, PA (US); Aaron Greenblatt, Jersey City, NJ (US)

(21) Appl. No.: 16/590,241

(22) Filed: Oct. 1, 2019

Related U.S. Application Data
(63) Continuation of application No. 16/542,106, filed on Aug. 15, 2019.
Application No. 16/542,106 is a continuation of application No. PCT/US2018/033005, filed on May 16, 2018.
(60) Provisional application No. 62/739,817, filed on Oct. 1, 2018.
(60) Provisional application No. 62/506,970, filed on May 16, 2017.

(51) Int. Cl. G06Q 30/02; G16H 20/10; G06Q 40/08

(52) U.S. Cl. CPC G06Q 30/0223; G06Q 20/387; G06Q 40/08; G16H 20/10

(57) ABSTRACT

In accordance with some embodiments, an alternative system for processing a request for a healthcare service is described. In accordance with some embodiments, the system obtains a diagnostic code and identifies one or more healthcare services, healthcare service providers and/or healthcare service facilities based on the diagnostic code and one or more additional data (e.g., a geographic location of a consumer and one or more terms of the consumer's healthcare benefit policy). The system may then output one or more offers to the consumer, at least one of the offers defining a reward to be provided to the consumer for selecting a healthcare service provider defined by the offer.

### CLAIM OF PRIORITY

The present application claims the benefit of U.S. Provisional Application No. 62/739,817 filed on Oct. 1, 2018 in the name of Peysekhman et al. and titled SYSTEM FOR FACILITATING HEALTHCARE EXPENDITURES. The present application is also a Continuation-in-Part of U.S. application Ser. No. 16/542,106 filed on Aug. 15, 2019 in the name of Gambill et al. and titled SYSTEM FOR FULFILLMENT OF PHARMACEUTICAL PRESCRIPTIONS, which Application is a Continuation Application of PCT Application No. PCT/US18/33005 filed on May 16, 2018 in the name of Gambill et al. and titled SYSTEM FOR FULFILLMENT OF PHARMACEUTICAL PRESCRIPTIONS, and which PCT Application claims the benefit of U.S. Provisional Application 62/506,970 filed on May 16, 2017 in the name of Gambill et al. and titled PHARMACY TRANSACTIONS FACILITATION SYSTEM AND METHOD. The entirety of each of the foregoing applications is incorporated by reference herein for all purposes.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Pharmaceutical prescription costs and other healthcare costs have been dramatically increasing over the past many years and are expected to continue to do so. Even persons covered by a pharmacy benefit insurance plan (also referred to as a prescription plan herein) and/or other health benefit insurance plan continue, year-over-year, to pay increasingly higher prices for each prescription they fill, doctor's visits, medical procedures and other health-related needs. Prescription and other healthcare costs continue to increase for reasons such as the co-pay amounts escalating, prescriptions not being covered by pharmacy benefit plans or counting towards an insured's out-of-pocket portion of the plan or particular healthcare service providers or treatment options corresponding to increasing prices even within a given healthcare or pharmacy benefit plan network.

One reason for the high cost of prescription drugs is that the savings that result from rebate or discount agreements that are entered into between prescription drug manufacturers and Pharmacy Benefit Managers (“PBMs”) are not typically passed on to the plan holders/consumers. PBMs are third party administrators of pharmacy benefit plans that work with pharmacies, pharmaceutical drug manufacturers and pharmaceutical benefit plan providers to negotiate discounts and rebates for prescription drugs and process claims for prescription drug claims and reimburse pharmacies for prescriptions filled by the pharmacies. Unfortunately, the end user or consumer that ultimately purchases the prescription drug often does not realize the benefits of any discounts negotiated with the retailers or rebates negotiated with the drug's manufacturer. Further, PBMs often add significant spreads to the cost of generic prescription drugs; in Applicant's estimation, generic drugs are marked up nearly 600% on average from the time they leave the manufacturer's premise until the time they are reimbursed by some combination of plan sponsor and plan member. Applicant has recognized that there is a long-standing need for a prescription drug redemption system that results in lower prescription drug prices to ends users or consumers and that works within the framework of the long-standing systems and processes for how prescription drugs are sold by pharmacies and for which pharmacies are reimbursed after filling a prescription. Lower prescription drug costs to consumers will result in various benefits, such as a greater adherence to a medical treatment plan (since many consumers report that the primary reason for not filling or re-filling a prescription is the high cost to the consumer when filling the prescription) and cost savings to employers or other health plan providers.

### BRIEF DESCRIPTION OF THE FIGURES

An understanding of embodiments described herein and many of the attendant advantages thereof may be readily obtained by reference to the following detailed description when considered with the accompanying drawings, wherein:

FIG. 1 is a block diagram of a system consistent with at least some embodiments;

FIG. 2 is a block diagram of a system consistent with at least some embodiments;

FIG. 3A is a flow diagram of an example offer assembly process consistent with at least some embodiments;

FIG. 3B is a flow diagram of an example pharmacy selection sub-routine that is consistent with at least some embodiments;

FIG. 3C is a flow diagram of an example price determination sub-routine that is consistent with at least some embodiments;

FIGS. 4A through 4H are example graphical user interfaces that may be output to a consumer in accordance with some embodiments; and

FIG. 5 is a flow diagram of an example electronic prescription card process that is consistent with at least some embodiments.

FIG. 6 is a flow diagram of an example process for providing to a consumer choices for different healthcare service providers based on geographical area.

FIG. 7 is a flow diagram of an example process for providing to a consumer choices for different treatment options or healthcare service facilities based on a diagnosis code from a healthcare service provider.

FIGS. 8A and 8B are examples of a graphical user interface that may be output to a consumer in accordance with some embodiments.

### DETAILED DESCRIPTION

The filling of prescriptions by consumers is currently a stressful and opaque process for the consumers (the persons for whom the prescriptions are provided by healthcare providers). The costs to consumers and pharmacy benefit providers (whether these be employers, corporate employers, health plans, labor unions, government entities and other organizations, collectively “Pharmacy Benefit Plan Providers” or PBPPs herein; also sometimes collectively referred to as “employers” herein) has been rapidly and continually increasing over many years. In accordance with some embodiments, PBPPs may also include employers or other healthcare benefit plan providers that provide healthcare benefits other than pharmacy benefits (e.g., healthcare provider benefits, treatment or diagnostic test benefits, etc.).

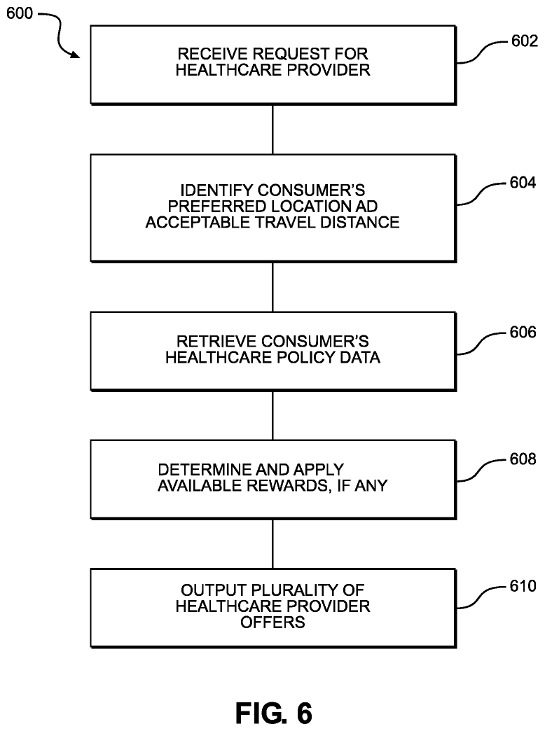

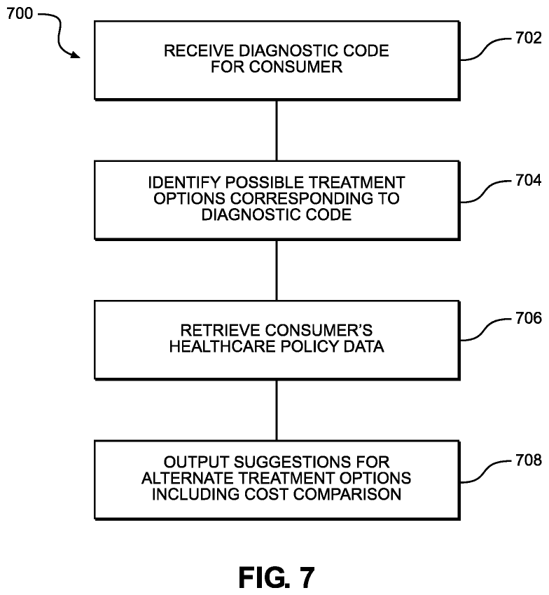

The pricing of pharmaceutical drugs (in terms of the price the consumer ends up paying for obtaining the drug, referred to as a Consumer Price herein) is a convoluted process that involves confidential contracts between the various pharmacies that fill the prescriptions and the Pharmacy Benefit Managers (PBMs) that administer pharmacy benefit plans on behalf of PBPPs, with the result being that the same pharmaceutical drug can have different Consumer Prices at different pharmacies. Under current practices, neither consumers nor PBPPs have ready access to the pricing information for different available pharmacies such that they can effectively accept and lock in a price for filling the prescription drug at a first pharmacy and thus realize the potential cost savings of having a prescription filled at the first pharmacy rather than a second pharmacy. Similarly, selecting a healthcare service provider, facility or treatment option can turn into a stressful process in which consumers are surprised at their out-of-pocket costs for selecting a particular healthcare service provider, facility or treatment option and often make such a choice without being informed of less costly alternatives (and without having a reasonably mechanism for obtaining and comparing alternatives). Although many of the embodiments and examples described herein are in the context of prescriptions and pharmacies, it should be noted that similar processes, systems and interfaces may be implemented for other types of healthcare costs in order to provide consumers with alternative choices to less costly options for healthcare service providers, facilities and treatment options (as described with respect to FIGS. 6 and 7 herein), in addition to or in lieu of providing an alternative prescription drug fulfillment system.

It should be noted that under the current prescription drug fulfillment system that involves PBMs managing and facilitating the fulfillment of prescriptions as a middleman between the PBPP, the consumer and the pharmacies, there are different monetary values exchanged between the various involved entities, which further obfuscates to the consumer whether he/she could be paying less for the prescription. The various monetary amounts involved in the fulfillment of a prescription are described below, for purposes of background information that sets forth the need for the invention(s) described herein.

A first monetary value involved in the fulfillment of a prescription is the monetary value a pharmacy has paid for obtaining the pharmaceutical drug (e.g., directly from the drug manufacturer or from a wholesaler) such that it has it in inventory and available for fulfillment of prescriptions. The monetary value that a pharmacy has paid for obtaining a pharmaceutical drug (whether a generic or brand version) is referred to as a Drug Cost herein.

A second monetary value involved in the fulfillment of a prescription is the monetary value a pharmacy is paid by a PBM upon filling the prescription. The PBM that administers the consumer's pharmacy benefit plan on behalf of the consumer's PBPP has different pricing agreements with different pharmacies (e.g., WALMART™ vs. CVS™). A pricing agreement between a pharmacy company and a PBM (often referred to as a Pharmacy Participation Agreement, or “PPA” herein). For generic drugs, the pricing agreement typically defines the parameters and aggregate discounts rates that are used to develop the respective Maximum Allowable Cost (“MAC”) for each generic pharmaceutical drug that the PBM will reimburse the pharmacy upon fulfillment of a prescription. Typically, MAC lists are published monthly and show the current MAC value for each generic pharmaceutical drug, per PBM. Sometimes, a pharmacy is reimbursed less than the MAC Pharmacy Reimbursement Amount for the various pharmaceutical drugs dispensed by the pharmacy and for which the pharmacy is reimbursed by the PBM. A Pharmacy Reimbursement Amount for a particular pharmaceutical drug, as the term is used herein unless indicated otherwise, is the monetary value a particular pharmacy is paid by a particular PBM (or the system of the present invention, as described herein) upon fulfillment of a prescription for that pharmaceutical drug (whether it is a MAC list price or otherwise and likely includes a fixed fulfillment fee per prescription). It should be noted that in many cases there may be a brand version and/or multiple generic versions that are suitable for fulfillment of a particular prescription, each such generic version (and the brand version) is considered a respective pharmaceutical drug for purposes of the present description and has a respective Pharmacy Reimbursement Amount associated therewith (sometimes multiple Pharmacy Reimbursement Amounts if different pharmacies have negotiated different Pharmacy Reimbursement Amounts for the same pharmaceutical drug). It should be noted that a pharmacy may receive other payments in exchange for filing a prescription (e.g., a transaction fee for each prescription filled). Further, if the consumer has a co-pay associated with his/her pharmacy benefit plan, the pharmacy may also collect this co-pay, which may be forwarded to the PBM by the pharmacy or credited against the Pharmacy Reimbursement Amount owed to the pharmacy by the PBM.

A third monetary value involved in the fulfillment of a prescription is the monetary value paid by the consumer's PBPP or employer to the PBM. Each PBM has a pricing schedule as part of its agreement with the PBPP or employer of the consumer, the pricing schedule defining a respective PBM Price for each pharmaceutical drug. A PBM Price, as the term is used herein, is the price a PBPP has to pay to a PBM once the PBM facilitates the fulfillment of the corresponding prescription drug at a particular pharmacy. Often, the PBPP or employer does not know what the Pharmacy Reimbursement Amount is set at as between a pharmacy and the PBM for a given pharmaceutical drug, it only knows the PBM Price for the drug as per its pricing schedule with the PBM. Typically a PBM profits significantly by including a substantial mark-up between the PBM Price of a particular pharmaceutical drug (i.e., what the PBM charges for the drug to the PBPP) and the Pharmacy Reimbursement Amount for the drug (i.e., what the PBM pays to a pharmacy for that drug). Depending on the PBMs pricing contracts with different pharmacies, that price differential can vary significantly. A PBM may further profit by collecting rebates from a drug manufacturer of a pharmaceutical drug (this being particularly true for brand drugs) but not passing any, or most, of that rebate on to the PBPP (e.g., in the form of a reduced PBM Price).

A fourth monetary value involved in the fulfillment of a prescription is the monetary value a consumer ultimately pays for filling a prescription for a pharmaceutical drug is referred to as a Consumer Price herein. A Consumer Price may, in some embodiments, be a co-pay amount (e.g., after a deductible threshold is met for the consumer's pharmacy benefit plan), the PBM Price (e.g., before a deductible threshold is met for the consumer's pharmacy benefit plan) or another amount. In the current complex and obfuscated environment of prescription fulfillment, consumers end up paying for a pharmaceutical drug based on the PBM Price of the drug (e.g., directly, in the deductible term of their pharmacy benefit plan, or indirectly even after their deductible amount is met because co-pays and premiums are calculated partially on how much the PBPP is expecting to pay to its PBM). Due to the lack of transparency inherent in the conduct of the PBMs, neither consumers nor PBPPs realize full the benefits of any rebates and fees the PBM may be receiving from the drug manufacturer or the potential for savings as a result of filling the prescription at a pharmacy that has a lower Pharmacy Reimbursement Amount than a pharmacy that has a relatively higher Pharmacy Reimbursement Amount.

Applicant's invention(s) deduces the Pharmacy Reimbursement Amounts as between different PBMs and different pharmacies by analyzing a variety of data sources, as well as estimated rebates available to PBMs from brand pharmaceutical drug manufacturers, and leverages the generated pricing data for the benefit of both consumers and PBPPs, with the result being not only savings to the consumer and PBPP but also, in many circumstances, additional rewards being earned by the consumer. Applicant's invention(s) provides an alternative to the conventional PBM model by providing an alternate mechanism that runs parallel to the PBM process (or as an alternate to the PBM process, for PBPPs or for consumers who may not have a pharmacy benefit plan).

In accordance with some embodiments, the invention(s) described herein outputs to a consumer who has been prescribed a pharmaceutical treatment (i.e., a prescription that can be filled with a brand or either a brand or at least one acceptable generic version of a drug, according to an acceptable formulary) a menu of offers, each offer defining a pharmacy location at which the prescription may be filled and a Consumer Price for each such location. The Consumer Price for each pharmacy location, in accordance with some embodiments, is based on the Pharmacy Reimbursement Amount for that pharmacy location (i.e., not on the PBM Price, which in the vast majority of circumstances will be significantly higher than the Pharmacy Reimbursement Amount).

In accordance with some circumstances (e.g., after a deductible threshold for the consumer's pharmacy benefit plan is met), a Reward Amount to be provided to the consumer may also be defined by one or more of the offers. A Reward Amount may be offered in order to incentivize the consumer to either use the system described herein (which system is referred to as the Alternate Pharmacy Benefit Manager system (“APBM” system)) rather than the traditional PBM's mechanism when the APBM system is running in parallel with the PBM and its prices are lower, or fill the prescription at a pharmacy location that, while less convenient for the consumer, has a relatively lower Pharmacy Reimbursement Amount and will thus be less costly for the consumer's PBPP. The APBM system(s) described herein provide an alternative to PBPPs as well as to consumers, by inserting itself as an alternate middleman (alternate to the PBM) between the PBPP and the pharmacy. In accordance with embodiments described herein, the APBM system allows the PBPP to only pay the Pharmacy Reimbursement Amount for prescriptions filled thereon (e.g., in exchange for the PBPP paying the APBM a relatively nominal fee (e.g., a per member per month fee or a per transaction fee) to the system) such that the PBPPs may realize the benefits of a consumer filling a prescription at a pharmacy that has a relatively lower Pharmacy Reimbursement Amount for the pharmaceutical drug with which the prescription is filled. The APBM system further allows for the consumer to realize these benefits for offering to the consumer a Reward Amount that is calculated or based on the differential between a PBM Price the PBPP would otherwise have to pay for the fulfillment of the prescription if the prescription fulfillment were to go through the conventional PBM process and the Pharmacy Reimbursement Amount. In accordance with some embodiments, the APBM system may provide the PBPP a choice as to what percentage or portion of this differential to include as a Reward Amount to the consumer (in other embodiments, this variable may be adjusted or set by the APBM, in some cases on a dynamic, transaction-by-transaction, customer-by-customer basis).

In accordance with some embodiments, a PBPP contractually agrees to provide APBM services to its plan members/consumers and in doing so provides specific information on its plan members and existing PBM plan to the APBM (e.g., a plan identifier). A consumer registers with an APBM prior to attempting to fill a prescription via the APBM system. The APBM system is thus able to obtain, update and utilize the specific information of a consumer's pharmacy benefit plan based on information obtained from the PBPP (e.g., co-pay amounts, deductible period and amount information, other consumers covered under the plan, the PBPP and PBM associated with the plan) when calculating and including the various data included in each offer output to the consumer (e.g., the Reward Amount and the Consumer Price).

In accordance with some embodiments, each offer further defines a Consumer Price for the pharmaceutical drug defined by the offer, which is the price the consumer is to pay for the pharmaceutical drug if the prescription is filled via the APBM system. The Consumer Price may comprise, for example: (i) a co-pay amount (e.g., if the prescription is being filled after a deductible period of the consumer's pharmacy benefit plan); (ii) a price based on the Pharmacy Reimbursement Amount (e.g., if the prescription is being filled during a deductible period of the consumer's pharmacy benefit plan); or (iii) the lower of the co-pay amount or the Pharmacy Reimbursement Amount of a given pharmacy location. In some embodiments, the menu of offers may also indicate to the consumer the PBM Price the consumer would pay if he/she were to fill the prescription via the traditional PBM route rather than using the APBM system (e.g., if the consumer is filling the prescription during a deductible period of their pharmacy benefits plan).

In accordance with some embodiments, once the consumer accepts one of these offers and commits to filling the prescription at the pharmacy location defined by the accepted offer, the consumer is guaranteed the Consumer Price defined by that offer (e.g., even if the APBM ends up having to pay a higher price to the pharmacy for the fulfillment of the consumer's prescription, as described in more detail below). In accordance with some embodiments, the consumer pays the Consumer Price to the APBM system, which then forwards that payment to the appropriate entity (e.g., the pharmacy location at which the prescription is filled). Thus, the consumer does not need to provide any payment to the pharmacy when filling the prescription at the pharmacy location defined by the offer.

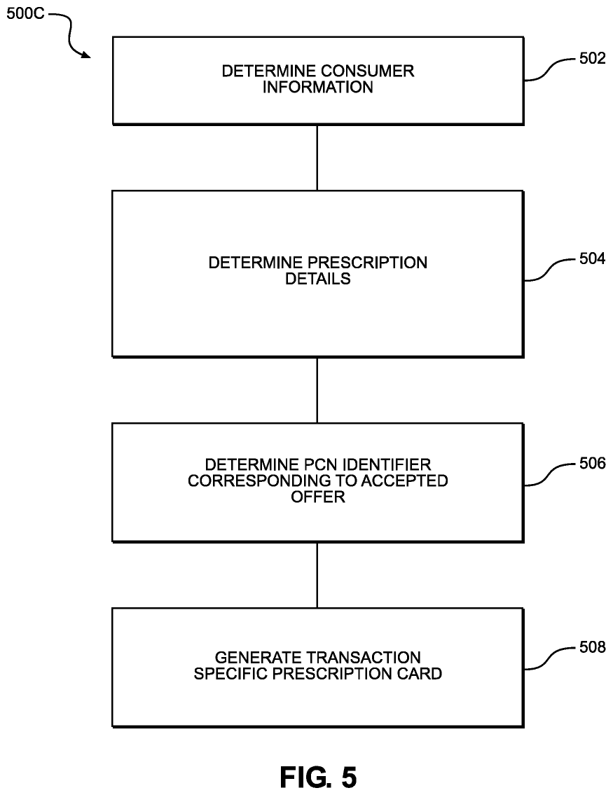

In accordance with some embodiments, the consumer is presented with the menu of offers, and able to accept an offer, via an app that is downloaded to the consumer's mobile device. In accordance with some embodiments, once the consumer accepts an offer, the APBM system (e.g., via the app on the mobile device) generates a digital code or electronic prescription code that the consumer can then present to the pharmacy location (e.g., in digital form on his/her mobile device or via a printed version thereof) in order to complete the transaction for his/her prescription (e.g., as described with respect to process 500 of FIG. 5).

Previous attempts at reducing the costs of prescription fulfillment have not addressed the consumer's need for a guaranteed price or price that can be relied upon, nor have they provided an opportunity for the PBPP and consumer to earn rewards and share in the cost savings when a consumer chooses to redeem a prescription at a pharmacy with a relatively low PBM price. Further, previous attempts at reducing the costs of prescription fulfillment have not been integrated with, or taken into account, a consumer's pharmacy benefit plan. Additionally, previous solutions have not been integrated with a claim processing system used by pharmacies to approve and process prescriptions, which allows a guaranteed price to be offered to a consumer at the point of sale.

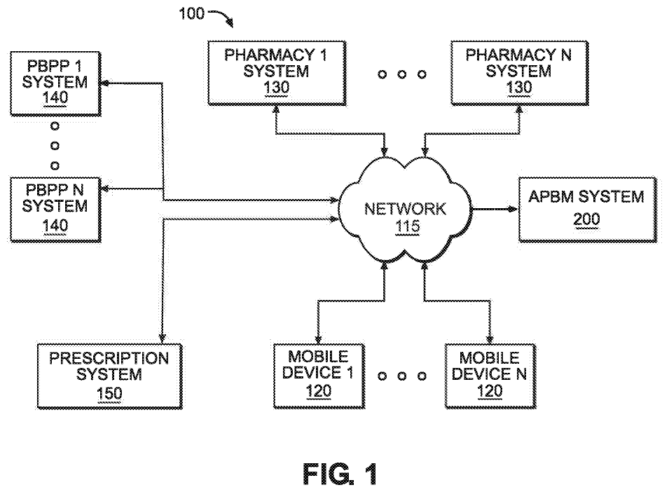

Referring now to FIG. 1, illustrated therein is a functional block diagram of an example system 100, which is consistent with some embodiments. The system 100 includes an APBM System which may, for example, by operated by or on behalf of an APBM in order to facilitate and/or manage the fulfilment of pharmaceutical prescriptions in accordance with at least some embodiments described herein. For example, the APBM System 200 may be operable to carry out one or more of the methods and/or functionalities described herein, such as (i) inferring, calculating or estimating, via one or more algorithms or protocols, the estimated Pharmacy Reimbursement Amount(s) for a particular pharmaceutical drug available via various pharmacies; (ii) identifying and maintaining an indication of PBM prices for various pharmaceutical drugs; (iii) maintaining one or more formularies (a formulary being a schedule of which pharmaceutical drugs are acceptable substitutes for one another or a listing of pharmaceutical drugs that are approved to be prescribed under a particular pharmacy benefit plan); (iv) identifying the estimated rebate amount available for one or more brand pharmaceutical drug; (v) obtaining and maintaining account information for member consumers (persons who register with the APBM in order to receive offers for prescription fulfillment options), such information including pharmacy benefit plan information for each such consumer; (vi) generating offers in response to a consumer entering prescription data; maintaining and updating information on offers accepted by consumers (e.g., including a redemption status of each such offer); (vii) facilitating the collection of payment from consumers; (viii) facilitating the provision of Pharmacy Reimbursement Amounts to pharmacies based on accepted and redeemed offers; and (ix) collecting payments from PBPPs (e.g., flat transaction fees for redeemed offers and/or payments of Pharmacy Reimbursement Amounts that have been paid, or that need to be paid, to pharmacies at which offers have been redeemed). In accordance with some embodiments, APBM System 200 may be operable to communicate, via a network 115, with (i) one or more mobile devices 120, devices of consumers or consumers participating in the APBM system described herein; (ii) one or more pharmacy computer systems 130; (iii) one or more PBPP systems 140; (iv) a commercial prescription claim processor 150 (e.g., ScriptClaim™ or an appropriate equivalent thereon, which stores prescription information such that it can be retrieved by a pharmacy upon a consumer requesting to fill the prescription at the pharmacy).

As will be appreciated by one skilled in the art, aspects of the present disclosure and of any of the components of the system 100 may be embodied as an apparatus that incorporates software, hardware, and/or firmware components. Any and all of the components of the system 100 may be implemented as a system controller, a dedicated hardware circuit, an appropriately programmed general-purpose computer, or any other equivalent electronic, mechanical, or electro-mechanical device. Any or all of the components of the system 100 may comprise, for example, one or more server computers operable to communicate with a plurality of computing devices (e.g., respective computing devices of PBPPs participating in the pharmaceutical prescription fulfillment program of the APBM and/or respective computing devices of one or more pharmacies participating in such program) and/or one or more additional devices such as a gateway server, router devices, or other devices for facilitating a pharmaceutical prescription fulfillment program as described herein.

The network 115 may comprise, for example, a mobile network such as a cellular, satellite, or pager network, the Internet, a wide area network, another network, or a combination of such networks. Although not shown in FIG. 1, other networks and devices may be part of system 100 and/or the network 1115 may comprise two or more networks operable to facilitate the routing of communications among the devices or components illustrated in FIG. 1. For example, in one embodiment, both the Internet and a wireless cellular network may be involved in routing communications and/or transmitting data among two or more devices or components illustrated in FIG. 1.

The communication between any of the components of system 100, whether via the network 115 or otherwise, may take place over one or more of the following: the Internet, wireless data networks, such as 802.11 Wi-Fi, PSTN interfaces, cable modem DOCSIS data networks, or mobile phone data networks commonly referred to as 3G, LTE, LTE—advanced, etc.

In some embodiments, additional devices or components that are not show in FIG. 1 may be part of a system for facilitating an alternate pharmacy prescription fulfillment program as described herein. For example, one or more servers operable to serve as wireless network gateways or routers may be part of such a system. In other embodiments, some of the functionality described herein as being performed by system 200 may instead or in addition be performed by a third party server operating on behalf of the system 200 (e.g., the APBM may outsource some functionality, such as registration of new consumers or managing the redemption of rewards earned by consumers). Thus, a third party server may be a part of a system such as that illustrated in FIG. 1. It should be understood that any of the functionality described herein as being performed by a particular component of the system 100 may in some embodiments be performed by another component of the system 100 and/or such a third party server. For example, one or more of the functions or processes described herein as being performed by APBM System 200 (e.g., a module or software application of the APBM System 200) or another component of system 100 may be implemented with the use of one or more cloud-based servers which, in one embodiment, may be operated by or with the help of a third party distinct from the APBM. In other words, while in some embodiments the APBM System 200 may be implemented on servers that are maintained by or on behalf of an APBM, in other embodiments it may at least partially be implemented using other arrangements, such as in a cloud-computing environment, for example.

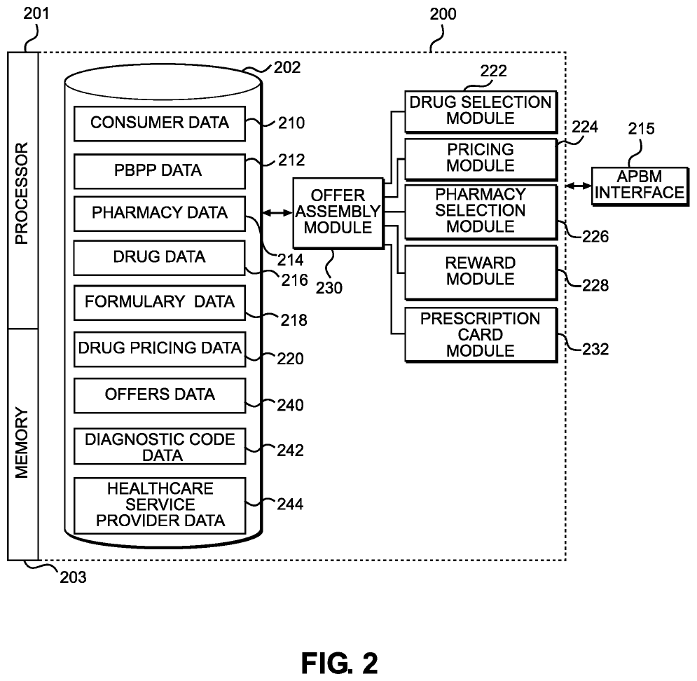

As also described herein, at least some of the embodiments described with respect to pharmacy benefit management or prescription drug fulfillment may also be adapted to facilitating the obtainment, by a consumer, of other health-related benefits, services or products. For example, as described with respect to FIG. 6, a system similar to System 100 (FIG. 1) and/or System 200 (FIG. 2) may be adapted to help a consumer obtain offers for different healthcare services, such as healthcare service providers (also referred to as healthcare providers herein) or healthcare facilities. In such an adaptation, the APBM System 200 may comprise a system operable to communicate with a plurality of healthcare service providers or facilities (e.g., in addition to being operable to communicate with a plurality of Pharmacy Systems 130). In another example, as described with respect to FIG. 7, a system similar to system 100 (FIG. 1) and/or System 200 (FIG. 2) may be adapted to provide to consumers offers for alternate medical treatment or diagnostic options. In such an adaptation, the APBM System 200 may further store in a database or other memory an indication of diagnostic codes that indicate a diagnosis a physician or other health service provider may utilize to indicate a diagnosis for a patient/consumer and at least one corresponding treatment option and/or diagnostic test that could or should be recommended for that diagnosis).

Turning now to FIG. 2, illustrated therein is an example of the APBM System 200 illustrated as being part of the system 100 of FIG. 1. The APBM System 200 may comprise, for example, a processor 201, a memory 203, a database 202 and a plurality of software modules 222-230.

In accordance with some embodiments, any or all of the components of the APBM System 200 may comprise one or more hardware components, such as microprocessors, microcontrollers, or digital sequential logic, etc., such as processor 201. The processor 201 may comprise one or more processors, such as one or more INTEL™ processors, working sequentially or in parallel. The processor 201 is in communication with, or operatively connected to, a memory 203. The memory 203 may comprise an appropriate combination of magnetic, optical, and/or semiconductor memory, and may include, for example, Random Access Memory (RAM), Read-Only Memory (ROM), a compact disc and/or a hard disk. The processor 201 and the memory 203 may each be, for example: (i) located entirely within a single computer or other device; or (ii) connected to each other by a remote communication medium, such as a serial port cable, telephone line or radio frequency transceiver. In one embodiment, the system 200 may comprise one or more devices that are connected to a remote server computer for maintaining databases.

The system 200 may further comprise a database 202, which may in some embodiments store data useful for implementing one or more embodiments described herein, non-limiting examples of which include (i) data associated with one or more consumers, (ii) data associated with one or more PBPPs; (iii) data associated with one or more pharmacies; (iv) data associated with one or more pharmaceutical drugs; and (iii) data associated with one or more offers generated by the APBM and accepted by a consumer (e.g., offers for which a consumer has paid the Consumer Price to the APBM). In accordance with some embodiments, the database 202 includes data, associated data structures, and database management software. The database 202 may, for example, be implemented using any well-known database management systems, including Microsoft SQL, Oracle, IBM DB2, etc. It should be noted that in some embodiments, database 202 (or at least some of the data described as being stored therein) may be stored in memory 203 and/or in another memory device accessible to the memory 203 and/or to processor 201. For example, in one embodiment database 202 (or at least some of the data described as being stored therein) may be stored in a memory of a third party server, such as a server of a cloud-based computing service with which the APBM may contract for purposes of storing data.

In some embodiments, the data described herein as being stored in database 202 may be stored across more than one database; the example data described herein as being useful in at least some embodiments is described as being stored in a single database 202 merely for purposes of simplicity. In accordance with some embodiments, one or more of the types of data 210-222 may be stored as a separate database (e.g., the pharmacy data 214 and the drug pricing data 218 may, in some embodiments, comprise a pharmacy database and a drug pricing database, respectively).

An example of a type of data that may be stored in database 202 includes consumer data 210 defining persons who have registered with the APBM in order to receive offers for fulfillment of pharmaceutical prescription. Such consumer data may include at least one of (i) information regarding the consumer's pharmacy benefit plan (if any), such as the co-pay amounts, deductible amount and current status, family members covered under the plan and pharmaceutical drugs covered under the plan; (ii) medical information of the consumer (e.g., allergies, medical conditions, other medications being taken); (iii) location information for selecting pharmacy locations to include in offers for the consumer (e.g., the consumer's home address or other preferred location, such as a work address); (iii) offers from the APBM accepted by the consumer (including redemption status thereof; alternatively this data may be stored in accepted offers data 232); (iv) payment information such as a preferred credit card to be charged for Consumer Prices of offers accepted by the consumer; and (v) rewards earned by the consumer based on accepted and/or redeemed offers (including redemption status of the reward(s); alternatively the rewards data may be stored separately). It should be noted that the information described herein as examples of the types of consumer data 210 may be stored in one or more related tables accessible to the APBM.

Another example of a type of data that may be stored in database 202 includes PBPP data 212 defining PBPPs that have agreed to participate in the pharmaceutical prescription fulfillment program of the APBM in accordance with at least some embodiments described herein. Such PBPP data may include at least one of: (i) an indication of the PBM utilized by the PBPP; (ii) a schedule of PBM prices the associated PBM charges to the PBPP; (iii) payment processing information (e.g., for processing invoices to the PBPP); and (iv) preferences of the PBPP (e.g., a preference for the percentage or portion of the differential between a PBM Price and a Pharmacy Reimbursement Amount to be provided to a consumer in the form of a reward).

Yet another example of a type of data that may be stored in database 202 includes pharmacy data 214 defining pharmacies that are available to be included in offers made by the APBM to consumers registered with the APBM. Such Pharmacy data may include, for one or more pharmacies, at least one of: (i) a geographical location of a pharmacy branch (e.g., to be utilized to calculate distance from a consumer's location, for purposes of selecting pharmacy locations to be included in offers made to consumers); (ii) a Pharmacy Reimbursement Amount for each of a plurality of pharmaceutical drugs available at the pharmacy, as contracted between the APBM and the pharmacy (e.g., in some embodiments the APBM may contract directly with partner pharmacies for specified Pharmacy Reimbursement Amounts for particular pharmaceutical drugs); (iii) a Pharmacy Reimbursement Amount for a plurality of PBMS for each of a plurality of pharmaceutical drugs available at the pharmacy (as it may be inferred or estimated by the PBM from various data sources, as described elsewhere herein); (iv) an indication of the particular generic version(s) of pharmaceutical drugs stocked by the pharmacy for a given medical condition or brand drug; (v) an indication of the Drug Cost to the pharmacy (which may be provided by the pharmacy to the APBM or inferred or deduced by the APBM from various sources, as described elsewhere herein); (vi) an indication of any incentives that the pharmacy is willing to offer to a consumer for selecting a particular pharmacy location; (v) an indication of payment processing information (e.g., for providing to the pharmacy the Pharmacy Reimbursement Amounts for prescriptions filled by the pharmacy in accordance with the terms of APBM offers accepted and redeemed by consumers).

Yet another example of the data that may be stored in database 202 is drug data 216 defining a list of prescription drugs and an indication of which generic pharmaceutical drugs are acceptable substitutes for one another or for a particular brand pharmaceutical drug. In one embodiment, the drug data may store the Generic Product Identifier (GPI) for each prescription drug. The GPI is a hierarchical classification system that comprises various data for prescription drugs in accordance with a particular code (e.g., the therapeutic class, dosage, strength, etc.). Other drug classification indicators that may be stored in drug data 216 include the American Society of Health-System Pharmacists (AHFS Drug Information) and First DataBank's Generic Sequence Number (GSN).

Yet another example of data that may be stored in database 202 is formulary data 218. In some embodiments, a plurality of formularies may be stored (e.g., different formularies for different pharmacy benefit plans). A formulary may comprise a list of the prescription drugs approved or covered under a particular pharmacy benefit plan or by a particular PBM and the respective consumer co-pay amounts relative to each drug.

Yet another example of the data that may be stored in database 202 is drug pricing data 220 defining one or more prices or monetary values corresponding to a particular pharmaceutical drug. For example, the drug pricing data may include, for each drug, at least one of: (i) a Drug Cost (e.g., per pharmacy); (ii) a manufacturer's retail price or suggested retail price; (iii) a Pharmacy Reimbursement Amount (e.g., per pharmacy, per pharmacy-PBM agreement and/or per pharmacy-APBM agreement; in some embodiments this may comprise the MAC list price for each drug); (iii) a PBM price (e.g., per PBM); and (iv) an estimated rebate amount or brand discount factor for brand pharmaceutical drugs (e.g., an estimation based on a protocols, algorithms and/or peer review processes as described elsewhere herein).

Another example of the data that may be stored in database 202 is offers data 240 that defines one or more offers that (i) a consumer has accepted (e.g., and for which a digital code or electronic prescription fulfillment card has been generated and provided to a consumer); (ii) has been offered or output to a consumer (even if the consumer has not accepted the offer(s)); and (iii) offers or prescriptions that a consumer has searched for). In some embodiments, some or all of such data (e.g., offers rejected by a consumer, prescriptions searched for by a consumer) may alternatively be stored in consumer data 210). In accordance with some embodiments, data on consumer search history and offers they did not accept may be utilized by the APBM system to tailor offers to the consumer (or other consumers) for purposes such as more appropriately sizing the rewards required to incentivize acceptance of certain offers.

In accordance with some embodiments, a new record may be opened and populated in the accepted offers data records each time a consumer accepts an offer output by the APBM. Such records may be accessed and updated, for example, upon receiving a confirmation from a pharmacy that a consumer has redeemed an offer (i.e., filled a prescription in accordance with the terms of the offer). Each record may indicate, for example, (i) details of the prescription (e.g., drug name, dosage and quantity); (ii) the name on the prescription (e.g. the consumer's name or a name of a family member of the consumer that is registered with the APBM); (iii) the pharmacy location defined by the offer that the consumer has accepted; (iv) an indication of the Consumer Price; (v) an indication of the cost of the prescription to the PBPP; (vi) an indication of the reward corresponding to the offer; (vii) a unique identifier identifying the offer; and (viii) some or all of the above corresponding to the offers the consumer did not select as a result of selecting this offer.

Another example of the data that may be stored in database 202 is diagnostic code data 242 defining one or more diagnostic codes that may be stored for reference by the system. The APBM system may utilize such diagnostic codes, for example, in identifying one or more of (i) appropriate service providers to include in offers output to a consumer (e.g., certain healthcare facilities and/or certain healthcare service providers may be stored as corresponding to, or qualified to provide, certain healthcare services corresponding to particular diagnostic codes); and/or (ii) alternate treatment options to output to the consumer (e.g., as described herein, certain diagnostic codes may correspond to various treatment options, with a hierarchy of which treatment options are most recommended, efficient or useful for certain diagnoses).

Yet another example of example of data that may be stored in database 202 is healthcare service provider data 244, defining one or more healthcare service providers or healthcare facilities that may be included in offers output to consumers or otherwise made available to consumers of the APBM. For example, the healthcare service provider data 244 may store, for a plurality of healthcare service providers or facilities, (i) address information; (ii) an indication of the services provided; and/or (iii) prices for the services provided (e.g., reimbursement price for each service, that the APBM would need to pay to the healthcare service provider if a consumer of the APBM were to obtain a service at the healthcare service provider or facility based on a recommendation, offer or interaction from/with the APBM). The APBM may utilize the data in the healthcare service provider data 244 to generate one or more offers for a consumer.

Thus, as described in accordance with some embodiments, an APBM function or feature may comprise offering one or more alternate options to consumers and PBPPs for fulfillment of pharmaceutical prescriptions or for obtainment of healthcare services that allows the PBPP and consumer to avoid the significant costs added to prescription fulfillment costs by PBMs, the confusing and non-transparent pricing system of obtaining healthcare services via conventional channels and/or to realize the benefits of relatively lower Pharmacy Reimbursement Amounts accepted by one pharmacy over another. The program takes into account a consumer's pharmacy benefit plan or other healthcare benefit plan (if any), lowers costs for the consumer as well as the PBPP and allows the consumer to be guaranteed a Consumer Price for a prescription or healthcare service upon accepting a corresponding offer even if the APBM is required to reimburse a pharmacy or healthcare service provider a higher price upon redemption of the offer.

The APBM program, in accordance with at least some embodiments, infers or determines otherwise confidential Pharmacy Reimbursement Amounts contracted as between pharmacies and PBMs (or, in some embodiments, negotiates low Pharmacy Reimbursement Amounts directly with PBMs) and eliminates PBMs as a costly middleman in a pharmaceutical prescription fulfillment while maintaining the profits of pharmacies at an acceptable level and working within the parameters of a consumer's existing pharmacy benefit plan.

It should be noted that the examples provided herein of what type of information may be included in which type of example data should not be taken in a limiting fashion. Modifications can be made as to what type of information is stored with which type of data, different types of data may be combined, some information may be stored with more than one type of data, etc.

The system 200 may further comprise one or more software module(s) for directing the processor 201 to perform certain functions. In accordance with some embodiments, software components, applications, routines or sub-routines, or sets of instructions for causing one or more processors to perform certain functions may be referred to as “modules”. It should be noted that such modules, or any software or computer program referred to herein, may be written in any computer language and may be a portion of a monolithic code base, or may be developed in more discrete code portions, such as is typical in object-oriented computer languages. In addition, the modules, or any software or computer program referred to herein, may in some embodiments be distributed across a plurality of computer platforms, servers, terminals, and the like. For example, a given module may be implemented such that the described functions are performed by separate processors and/or computing hardware platforms.

With reference to FIG. 2, it should be understood that any of the software module(s) or computer programs illustrated therein may be part of a single program or integrated into various programs for controlling processor 201. Further, any of the software module(s) or computer programs illustrated therein may be stored in a compressed, uncompiled, and/or encrypted format and include instructions which, when performed by the processor 201, cause the processor 201 to operate in accordance with at least some of the methods described herein. Of course, additional and/or different software module(s) or computer programs may be included and it should be understood that the example software module(s) illustrated and described with respect to FIG. 2 are not necessary in any embodiments. Use of the term “module” is not intended to imply that the functionality described with reference thereto is embodied as a stand-alone or independently functioning program or application. While in some embodiments functionality described with respect to a particular module may be independently functioning, in other embodiments such functionality is described with reference to a particular module for ease or convenience of description only and such functionality may in fact be a part of integrated into another module, program, application, or set of instructions for directing a processor of a computing device.

According to an embodiment, the instructions of any or all of the software module(s) or programs described with respect to FIG. 2 may be read into a main memory from another computer-readable medium, such from a ROM to RAM. Execution of sequences of the instructions in the software module(s) or programs causes processor 201 to perform at least some of the process steps described herein. In alternate embodiments, hard-wired circuitry may be used in place of, or in combination with, software instructions for implementation of the processes of the embodiments described herein. Thus, the embodiments described herein are not limited to any specific combination of hardware and software. Some non-limiting examples of software module(s) that may be utilized in system 200 include: (i) a drug selection module 222; (ii) a pricing module 224; (iii) a pharmacy selection module 226; (iv) a reward calculation module 228; (v) an offer assembly module 230; and (vi) a prescription card module 232.

In the example embodiment illustrated in FIG. 2, offer assembly module 230 is illustrated as communicating with (i) a drug selection module 222; (ii) a pricing module 224; (iii) a pharmacy selection module 226; (iv) a reward module 228; and (v) a prescription card module 232. Any of (i) a drug selection module 222; (ii) a pricing module 224; (iii) a pharmacy selection module 226; (iv) a reward module 228; and (v) a prescription card module 232 may be operable to communicate with the database 202 (either directly or via the offer assembly module 230) and thus able to access or retrieve various data therefrom. Such an arrangement is illustrated as one example of how the data in database 202 may be accessed, modified and utilized.

Generally, the modules 222-232 should be understood as being accessible to processor 201, irrespective of how in particular they are arranged within the system 200, to implement one or more embodiments described herein. As described, one or more of the modules 222-232 may be operable to utilize at least some of the data stored in database 202. Further, in accordance with some embodiments, one or more of the modules 222-232 may be operable to retrieve, manipulate, select, update, modify and/or determine data that is transmitted to and stored in database 102.

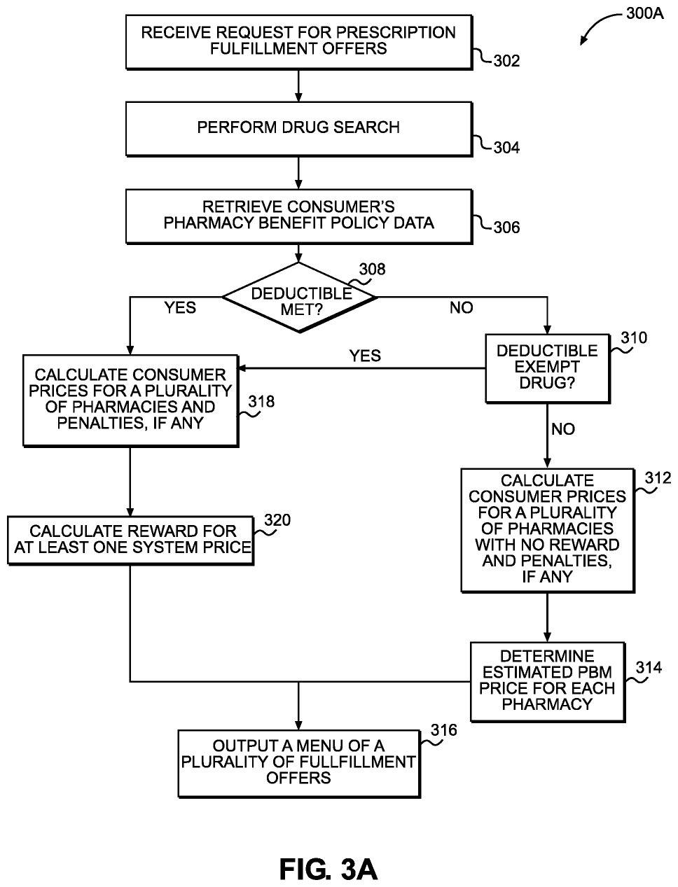

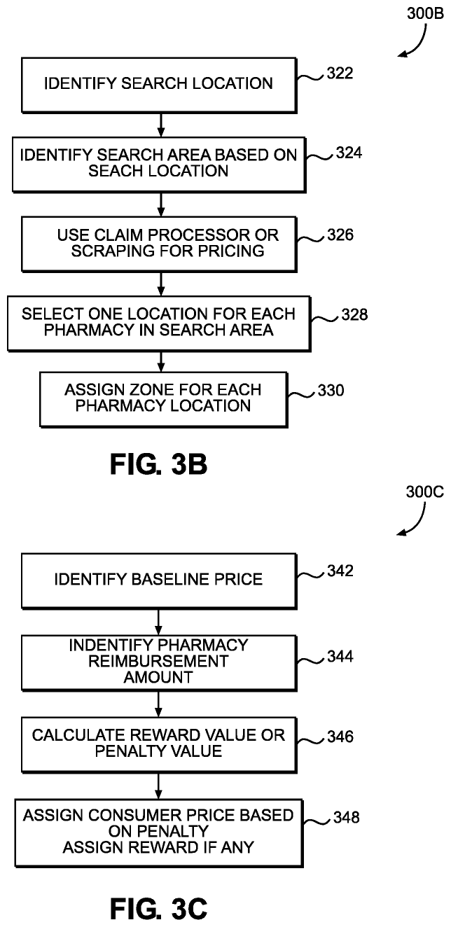

Offer assembly module 230 may, in accordance with some embodiments, operate to manipulate the data from database 202 in order to generate a plurality of offers to be output to a consumer via a mobile device 120 (FIG. 1). In accordance with one embodiment, the offer assembly module 230 or another component of system 200 may be operable to output the plurality of offers to a consumer (and, in some instances, receive input from a consumer, such as a request for a plurality of offers for filling a prescription, an acceptance of an offer and/or information regarding pharmacy benefit plan of the consumer) via an APBM user interface 215. The description herein of process 300 (described with respect to FIG. 3) provides some examples of how the modules 222-232 may be operable to utilize the data stored in database 202 to generate a plurality of offers (each offer defining the terms under which a given consumer may fill a given prescription at a specified pharmacy location).

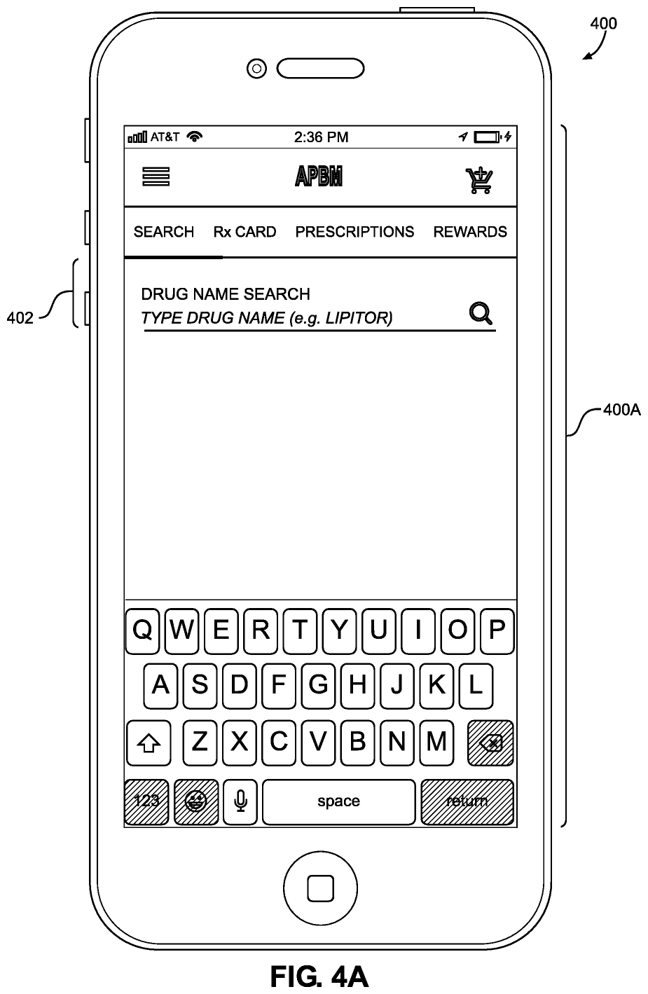

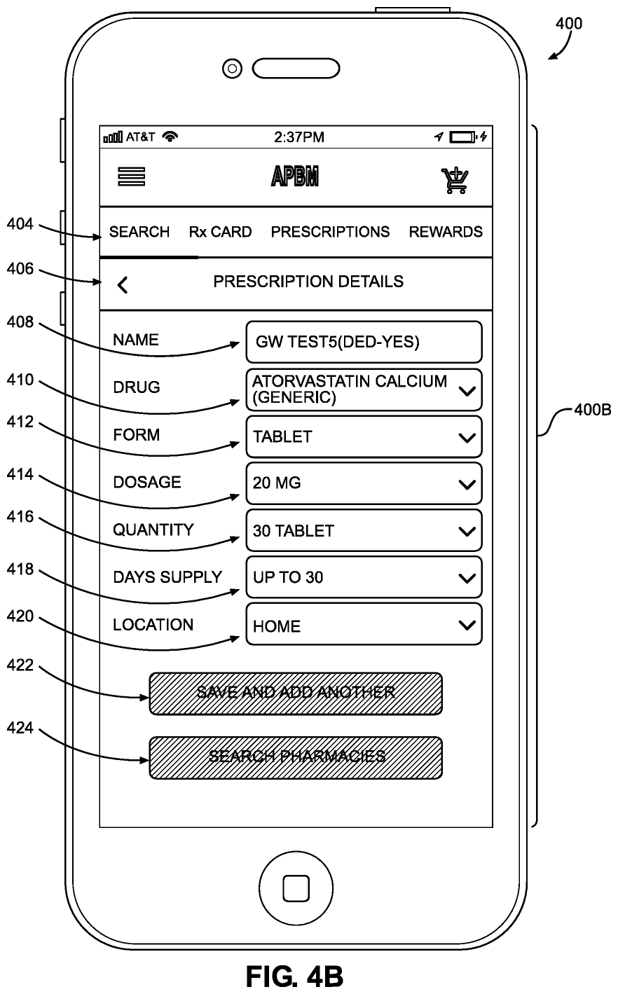

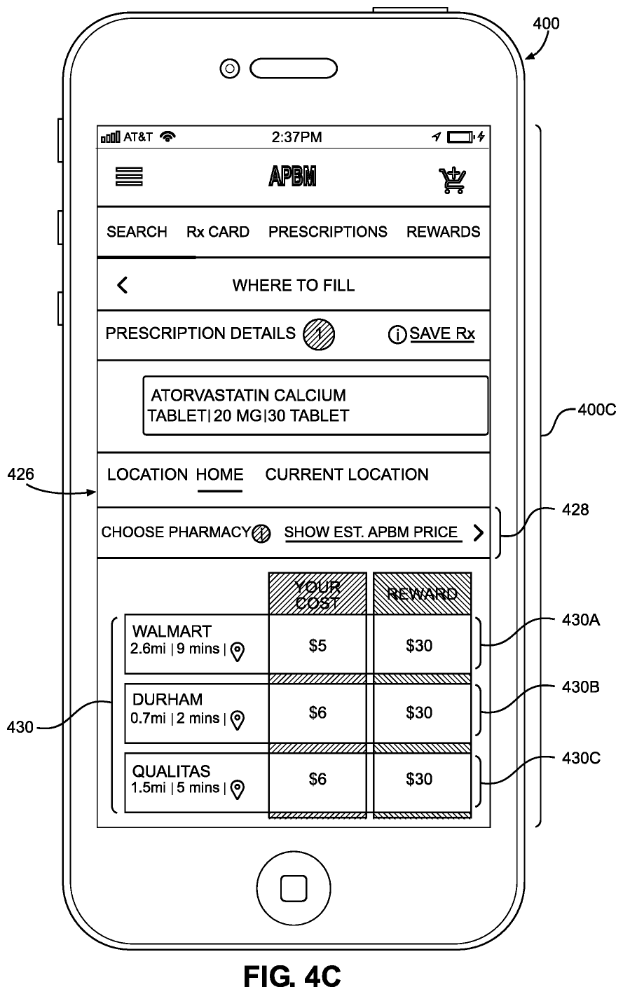

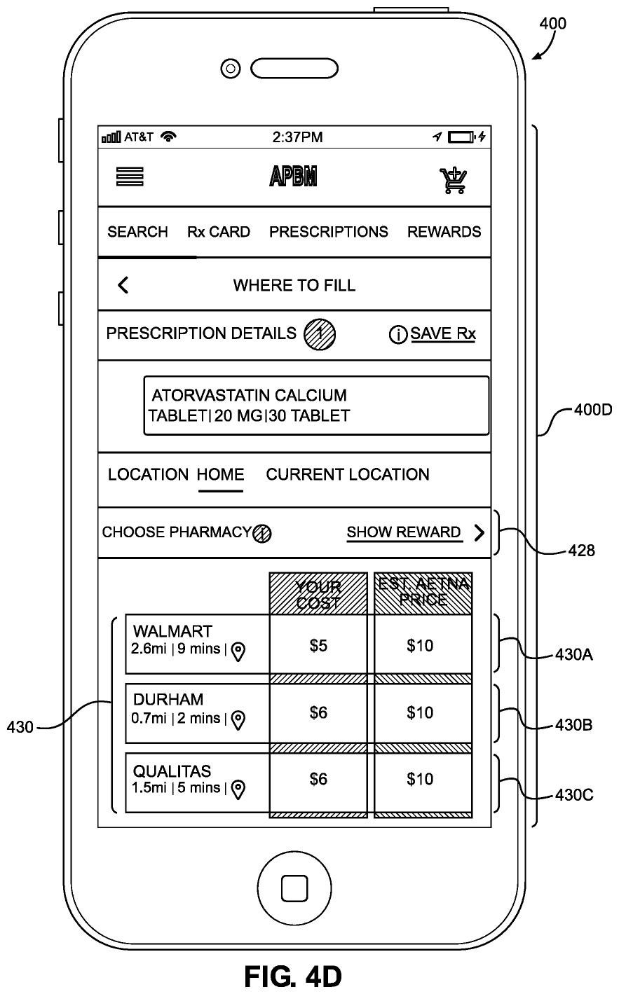

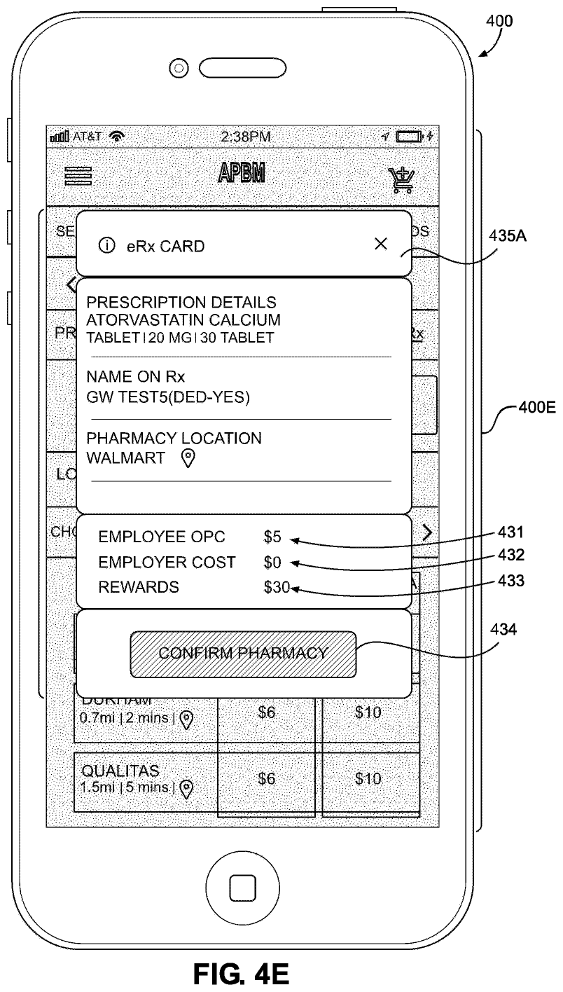

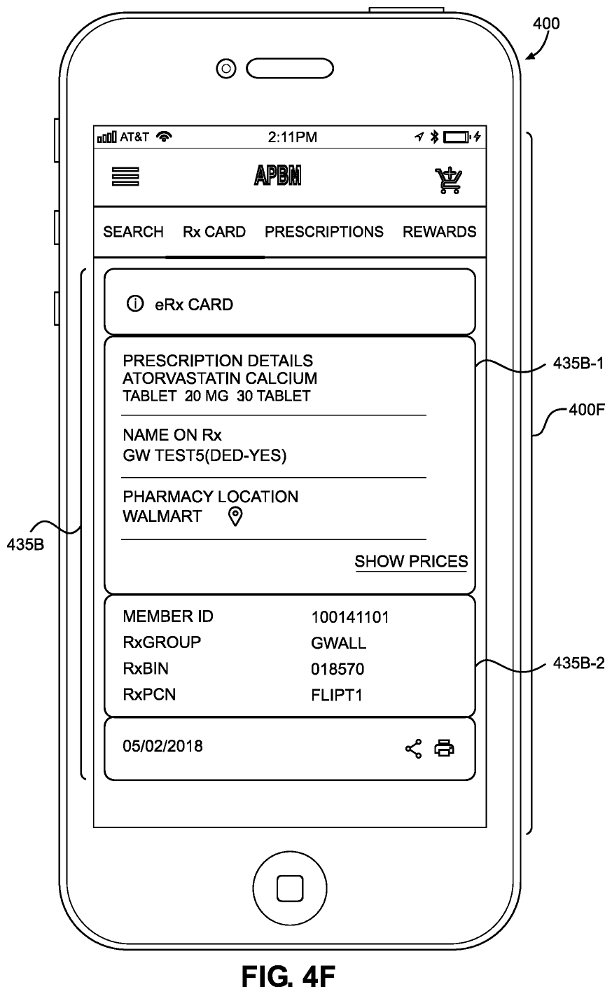

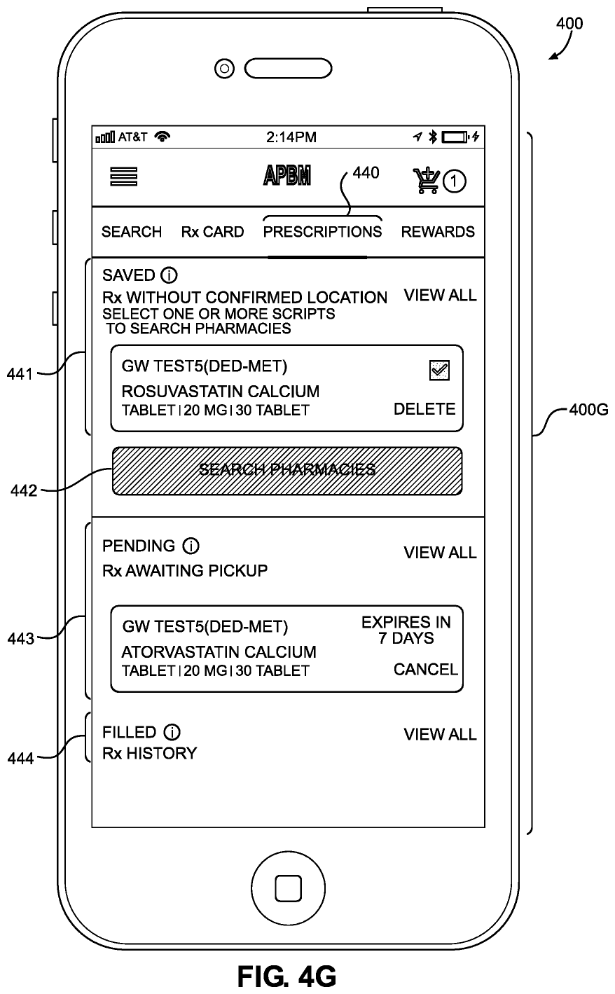

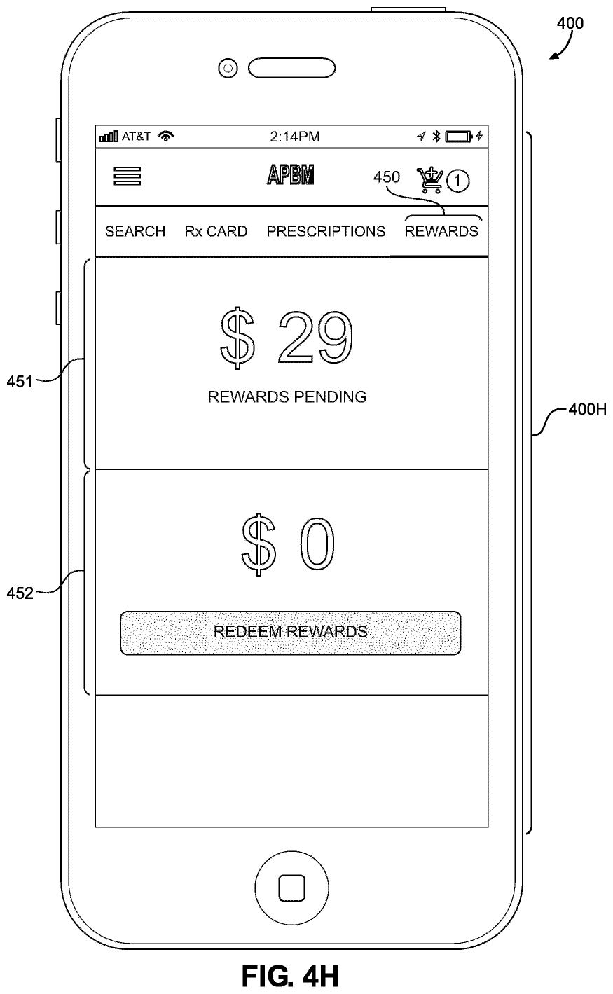

The APBM interface 215 may, for example, take the form of a Web server that conveys data in HTML, XML, or other well-known formats using well-known transmission protocols, such as HTTP and TCP/IP. Proprietary protocols and data formats may also be used. A mobile device 120, which may take the form of a desktop computer, laptop computer, personal digital assistant (PDA), mobile phone, smart phone, or other computing device, may be used by a consumer or consumer to obtain information relating to fulfillment of pharmaceutical prescriptions and such information may be presented to the consumer or consumer via the APBM interface 215. FIGS. 4A-4H illustrate non-limiting examples of the type of information that may be output to, and mechanisms which may be collected from, a consumer via an APBM interface such as APBM interface 215.

Turning now to FIG. 3A, illustrated therein is an example process 300A which provides one embodiment of how a plurality of offers for fulfilling a pharmaceutical prescription outside of the traditional PBM structure. Process 300A may, for example, be performed by APBM system 200 (FIG. 2). In some embodiments, the process 300A may be performed and/or implemented by and/or otherwise associated with one or more specialized and/or specially-programmed computers (e.g., the processor 201 of FIG. 2). It should be noted, with respect to process 300A and all other processes described herein, that not all steps described with respect to the process are necessary in all embodiments, that the steps may be performed in a different order in some embodiments and that additional or substitute steps may be utilized in some embodiments.

Process 300A will be described herein with reference to FIGS. 4A-4F, which illustrate a series of graphical user interfaces which may be presented to a consumer during a progression of the consumer's request for a plurality of offers for pharmacy locations at which a prescription may be filled, as well as examples of user interfaces that the consumer may access via an APBM app in order to track and manage his prescriptions and rewards. FIGS. 4A-4H each illustrate a respective graphical user interface (GUI) as it may be output on a mobile device 400 (which may comprise an example of a mobile device 120, FIG. 1). The GUI 400 may comprise several tabs or screens, as illustrated in each of FIGS. 4A-4H. Thus, FIG. 4A illustrates a screen 400A that may be output as part of an APBM GUI, FIG. 4B illustrates a screen 400B that may be output as part of an APBM GUI, FIG. 4C illustrates a screen 400C that may be output as part of an APBM GUI, FIG. 4D illustrates a screen 400D that may be output as part of an APBM GUI, FIG. 4E illustrates a screen 400E that may be output as part of an APBM GUI, FIG. 4F illustrates a screen 400F that may be output as part of an APBM GUI, FIG. 4G illustrates a screen 400G that may be output as part of an APBM GUI and FIG. 4H illustrates a screen 400H that may be output as part of an APBM GUI.

In accordance with some embodiments, the APBM GUI may be made available via a software application operable to receive and output information regarding pharmaceutical prescription fulfillment offers as generated and managed by an APBM in accordance with embodiments described herein. It should be noted that many variations on such graphical user interfaces may be implemented (e.g., menus and arrangements of elements may be modified, additional graphics and functionality may be added). The graphical user interfaces of FIGS. 4A-4H are presented in simplified form in order to focus on particular embodiments being described.

The FIGS. 4A-4F illustrate successive screens that may be output to a consumer from the time a consumer enters a name of a prescription drug and requests offers for fulfilling the prescription, as the offers are output to the consumer, the consumer confirms an accepted offer and an electronic or digital prescription confirmation or “card” is output to the consumer for the confirmed accepted offer. FIGS. 4G and 4H each respectively illustrate a screen that may be output to a consumer upon demand, as the consumer desires to view certain information regarding prescriptions or rewards he/she has with the APBM system.

Turning now to FIG. 3A, process 300A is initiated at block 302 upon it being determined that a request for prescription fulfillment offers has been received from a consumer (e.g., via an APBM interface, such as APBM interface 215 or such as illustrated in GUI 400A of FIG. 4A). In accordance with some embodiments, a consumer may enter prescription information for a new prescription while at a medical provider's office, such that the medical provider (e.g., doctor) can help the consumer provide the appropriate information when submitting the request (e.g., the drug name, dosage and quantity). For example, the consumer (who has pre-registered with the ABPM system) may log in to his account with the APBM using a software application pre-loaded onto his mobile device and select an option to enter a new prescription (e.g., as illustrated in the search bar 402 of FIG. 4A). In one embodiment, the consumer may enter the brand name of a prescription drug in order to initiate the process, while in other embodiments the consumer may provide a generic name or even a name that is descriptive of a class of drugs or a medical condition that the desired drug is approved to treat. The consumer may also be prompted to provide additional information for the prescription, such as the dosage, form (e.g., liquid or tablet) and quantity of the drug as being prescribed by the medical provider.

The APBM system then launches a drug search in block 304, based on the information received from the consumer in block 302 (e.g., based on the information provided in GUI 400A and/or GIU 400B). In accordance with some embodiments, the search for an acceptable pharmaceutical drug based on the information received from the consumer in step 302 may be performed by the drug selection module 222 of FIG. 2 and utilizing the data stored in database 202, such as the drug data 216, the formulary data 218 or other prescription drug data). In accordance with some embodiments, the APBM maintains a database of pharmaceutical drugs such that it can search for and retrieve acceptable substitutes for a drug name as entered by the consumer. For example, if the consumer has entered a brand name for a pharmaceutical drug, the APBM may be operable to look up any and all generic versions of the drug. In accordance with one embodiment, the system may utilize the Generic Product Identifier (GPI) classification system to identify acceptable generic equivalents. In one embodiment, the system may store a ranking of generic equivalents and alternatives for a brand drug and may be programmed to select the highest ranked generic equivalent or alternative. In one embodiment, a ranking of equivalents may be based on the Pharmacy Reimbursement Price corresponding to each equivalent (e.g., the lowest priced equivalent being ranked the highest). In some embodiments, the APBM may facilitate a proprietary crowd-sourcing or peer-review process via which medical professionals are polled to provide their professional opinions as to which prescription drugs are acceptable substitutes for one another. In the event that the consumer searched for a drug and at least one equivalent or alternative generic version was found, or cheaper alternative brand version was found, the system may suggest the alternative version to the consumer and/or the medical professional prescribing the drug and, if the consumer agrees to view offers for the alternative version, the system may proceed with process 300A using the alternative version (in accordance with some embodiments, the consumer may be provided with a mechanism to switch back to the brand drug if desired, in which case some of the following steps of process 300A may be repeated for the brand drug). If the consumer had entered a generic prescription drug name to initiate the search, the APBM system may look up the generic drug in its drug database (e.g., in drug data 216 of memory 202) to either find the closest match and present it to the consumer or to find with the highest ranked product equivalent.

In accordance with some embodiments, the APBM system may request that the consumer confirm details of the prescription for which fulfillment offers are being requested and/or verify the drug information (e.g., dosage, quantity or days of supply needed) prior to continuing to step 306. In some embodiments, confirming the details of the prescription may also include confirming the name of the patient named on the prescription for which fulfillment offers are being requested (e.g., the prescription may be for a child, spouse or other family member of the consumer who is submitting the request for the offers). Turning briefly to FIG. 4B, illustrated therein is an example screen 400B that may be output to a consumer, via which the consumer is requested to confirm information regarding the prescription for which fulfillment offers will be assembled. In the example screen 400, area 406 indicates to the consumer that the consumer is viewing details of the prescription (and allows the consumer to go back to a previous screen, such as screen 400A, by selecting the arrow in the left side of this area). Area 404 indicates a plurality of different types of information that the consumer can view using the software app. Area 408 indicates the name of the consumer who has logged in to the app and is requesting the prescription offers. In some embodiments, this area or another area may indicate the name of the patient named on the prescription, which may differ from the consumer who is requesting the offers (e.g., the patient may be a child or spouse of the consumer) and/or allow the consumer to indicate a name of a patient other than the consumer. Area 410 indicates the name of the prescription drug for which the prescription offers will be assembled. For example, area 410 may indicate the name of the drug as entered by the consumer in area 402 of screen 400A or the name of an alternate generic version of that drug that the system is recommending. In accordance with some embodiments, area 410 may comprise a drop-down menu via which the consumer may select alternate versions of the drug or other acceptable substitute drugs (e.g., as identified in step 304, which may in some embodiments comprise populating this drop-down menu). Area 412 indicates the form of the drug (e.g., tablet vs. liquid) that is going to be used to assemble the offers and allows the consumer (e.g., via a drop-down menu) to select an alternate form. Area 414 indicates the dosage of the drug that is going to be used to assemble the offers and allows the consumer (e.g., via a drop-down menu) to select an alternate form. Area 416 indicates the quantity of the drug that is going to be used to assemble the offers and allows the consumer (e.g., via a drop-down menu) to select an alternate form. Area 418 indicates the number of days of supply of the drug that is going to be used to assemble the offers and allows the consumer (e.g., via a drop-down menu) to select an alternate form. Area 420 indicates the search location that is going to be used to search for pharmacies at which the prescription may be fulfilled (the search location and process of searching for pharmacies is described in detail with respect to process 300B of FIG. 3B). In accordance with some embodiments, the content of any of the drop-down menus illustrated in areas 410-418 may be populated by the system, such as in step 304 based on the results of step 304. In some embodiments, data entry formats other than drop-down menus may be used in any of the fields 410-420 (e.g., the consumer may be allowed to enter data free-form or select options via radio buttons or pop-up windows). Once the consumer is satisfied with the prescription details shown, he/she can select area 424 to launch the offer assembly process (or to allow the offer assembly process to continue). Alternately, the consumer may select area 422 to save the details of the present prescription and continue to add information for another prescription, in which case the consumer may be taken to another screen or returned to screen 400A.

Returning now to FIG. 3, as described herein, a consumer who would like to see offers for prescription drug fulfillment options from the APBM system may download its software app onto his/her mobile device and register with the APBM system. In registering with the APBM system, the consumer may, in at least some embodiments, be asked to provide information on his/her pharmacy benefit plan or plan (or this information may be provided by the consumer's PBPP or employer). Such information may include, for example, the PBPP of the plan, a group number, a member identification number, a type or classification of the plan and the names of all persons covered under the plan (e.g., any family members of the consumer who are covered under the plan, their birthdates, etc.). The APBM system may be operable to verify the consumer's coverage under this plan (e.g., by contacting the PBPP directly). In some embodiments the APBM system may further be operable to obtain from the PBPP additional information regarding the consumer's benefits under the plan for use in assembling offers (e.g., the deductible amount, period and associated restrictions or conditions; the coverage period under the plan; the formulary to be utilized for the plan). Such information defining the consumer's pharmacy benefit pharmacy may be stored by the APBM (e.g., in the patient data 210 of database 202) and subsequently retrieved when the consumer inputs a request to receive fulfillment offers for a new prescription.

In accordance with some embodiments, the APBM may have relationships with certain PBPPs and only consumers who have plans from these PBPPs may be able to register with the APBM. In some embodiments, the APBM may receive (e.g., periodically) and store updated information regarding a consumer's pharmacy benefit plan (e.g., from the PBPP corresponding to the consumer's plan or another party), such as whether the consumer is still within the deductible period and/or how much is left in the consumer's deductible amount. In other embodiments, consumers may be able to register with the APBM system even if their PBPP does not have a relationship with the APBM. In some embodiments, consumers who do not have a pharmacy benefit plan or who do not desire to provide information regarding their pharmacy benefit plan to the APBM for purposes of having the APBM assemble prescription fulfillment offers may also be able to register with the APBM.

In block 306, the pharmacy benefit plan information corresponding to the consumer for whom offers are currently being assembled is retrieved. For example, a record for the consumer may be stored in a database of the APBM and accessed based on the login credentials the consumer entered when launching the software application or otherwise initiating a request for the offers.

The APBM system may retrieve information associated with the consumer's pharmacy benefit plan that will be used to assemble one or more prescription fulfillment offers. One example of information related to the consumer's pharmacy benefit plan that may be retrieved is an identification of the PBM associated with the PBPP that provides the plan to the consumer such that the appropriate formulary corresponding to the PBM may be retrieved from a database (e.g., from the formulary data 218 of memory 202, FIG. 2). In one embodiment, the PBM identification may be determined based on information stored in consumer data 210 of memory 202 while in other embodiments the PBM may be identified based on information stored in PBPP data 212 of memory 202. Other examples of information related to the consumer's pharmacy benefit plan that may be retrieved and utilized in assembling one or more prescription fulfillment offers include, without limitation, (i) an indication of whether the consumer is still within the deductible period of the plan; (ii) persons covered under the consumer's plan; (iii) an indication of any co-insurance or co-pay amounts the consumer is responsible for paying and corresponding rules or conditions that govern the application of such co-insurance or co-pay; (iv) other prescriptions prescribed to the same patient of the present prescription; (v) health-related information such as weight, age and medical treatment history; (vi) income from the company sponsoring the plan; (vii) zip code; (viii) length of employment with the company sponsoring the plan; and/or (ix) position within the company sponsoring the plan.

Block 308 is a decision block as to whether to proceed to block 310 or block 318, based on whether the consumer is currently within his/her deductible period (if any) or has met their maximum annual out of pocket (if any) the pharmacy benefit plan retrieved in block 306. In accordance with some embodiments, the calculation of a Consumer Price (the monetary value the consumer will pay out-of-pocket for the prescription drug of the present prescription) is calculated differently depending on whether the consumer is still under a deductible period (in which case the consumer will be expected, under most circumstances, to pay for the prescription drug without the PBPP paying for any portion of it) or whether the consumer is no longer under a deductible period (in which case, under most circumstances, the consumer will be responsible for paying the co-pay and a co-insurance amount (if any) as defined by his/her pharmacy benefit plan and the PBPP will be responsible for paying the remainder).

If it is determined in step 308 that the deductible amount for the consumer's pharmacy benefit plan has not yet been met (i.e., the answer to step 308 is “no”), the process 300A proceeds to block 310; otherwise the process 300A proceeds to block 318. In block 310 it is determined whether the prescription drug for which prescription fulfillment offers are currently being assembled is one that is exempt from the plan's deductible amount. Some pharmacy benefit plans define a list of drugs (e.g., preventative maintenance drugs like cholesterol and blood pressure medications) that are “deductible exempt” meaning that the consumer only has to pay the co-pay amount for these drugs even during the deductible period and the PBPP is responsible for the remaining cost. The system may access information associated with the consumer and/or the consumer's pharmacy benefit plan (e.g., consumer data 210, PBPP data 212 and/or formulary data 218 of memory 202, FIG. 2) to determine whether the drug is a deductible exempt drug under the consumer's pharmacy benefit plan. If it is determined that the drug is a deductible exempt drug, the process 300A continues to block 318; otherwise the process continues to block 312.

In some embodiments, a consumer who does not have (or has not provided to the APBM an indication of having) a pharmacy benefit plan may be registered with the APBM and be requesting prescription fulfillment offers. In such an embodiment, the process 300A may include a step that determines whether this is the case and performs a different subroutine or process as a result. Alternatively, such a consumer's offers may be assembled in accordance with process 300A but the system may be programmed to handle such a consumer in a specific manner at different blocks of process 300A (e.g., omit step 304 and determine the answers to each of step 306 and step 308 is “no”).

In block 312 consumer prices are calculated for a plurality of pharmacies at which the consumer may redeem the prescription, in a scenario in which the consumer has not yet satisfied his/her deductible and the prescription is for a drug that is not a deductible drug (or in a scenario in which the consumer does not have a pharmacy benefit plan and is relying on the APBM system as his primary prescription fulfillment mechanism). In this scenario, the consumer is responsible for the entire cost of the drug (i.e., the cost is not shared with a PBPP). As a preliminary sub-routine for determining the consumer price for each of a plurality of pharmacy locations may comprise a process for selecting the plurality of pharmacy locations, one for each offer being assembled. FIG. 3B illustrates one example process 300B for selecting a plurality of pharmacy locations to be included in offers being assembled for a prescription and will now be described.

Turning now to FIG. 3B, illustrated therein is an example process 300B which provides one embodiment of how a plurality of specific pharmacy locations, one for each of a plurality of offers that are being assembled for a prescription, may be selected. Process 300B may, for example, be performed by APBM system 200 (FIG. 2). In some embodiments, the process 300B may be performed and/or implemented by and/or otherwise associated with one or more specialized and/or specially-programmed computers (e.g., the processor 201 of FIG. 2). In one embodiment, the process 300B may be performed by a sub-routine of APBM 200, such as the pharmacy selection module 226 (FIG. 2).

As a preliminary matter, a search location for the prescription is identified at block 322. The search location may, in accordance with some embodiments, comprise a starting location for the search based on a location associated with the consumer who has requested the prescription fulfillment offers. For example, the search location may comprise one of: (i) a current location of the consumer's mobile device; (ii) a home address of the consumer; or (iii) a stored addressed that the consumer has provided as a preferred address to be used as a search location (e.g., the consumer's work address). In some embodiments, the consumer may enter or select (and update as desired) a default location to be used as a search location for his prescriptions and have this stored in association with his/her data by the APBM system (e.g., as part of the consumer data 210 in memory 202). In some embodiments, as illustrated in area 426 of the screen 400C illustrated in FIG. 4C, the GUI may output to the user a mechanism for toggling back-and-forth between two or more search location choices (in the example of FIG. 4C, this being a choice between the consumer's stored home address and a current location of the consumer's mobile device). In accordance with some embodiments, identifying the search location may comprise identifying the geocodes for, or geocoding, the search location. Geocoding is the computational process of transforming a postal address description to a location on the Earth's surface (spatial representation in numerical coordinates).

Once the search location is identified in block 322, a search area is determined in block 324. A search area is the geographical area around the search location within which potential pharmacy locations are to be searched. Although any scope and shape of a search area may be utilized and the embodiments described herein are not dependent on any particular search area scope or shape, in accordance with one embodiment the search area is identified by creating a box by adding ten (10) miles to the search location longitude and latitude in each direction.

In block 326, pricing information that may be useful for narrowing down the pharmacies within the search area that may be selected for inclusion in a plurality of prescription offers may be determined, in accordance with some embodiments (in other embodiments, step 326 may be omitted or different or additional filters may be applied to narrow down a list of possible pharmacies). For example, information from one or more of the following sources may be utilized to generate a list of potential pharmacies within the search area: (i) a claim processor; (ii) publicly available information from the world wide web (e.g., www.cms.gov); and (iii) a proprietary database such as the pharmacy data 214 of memory 202). Such information may be obtained by scraping it from one or more of the available sources or having it provided by the one or more sources to the APBM system. In some embodiments, additional information corresponding to the pharmacies (e.g., MAC prices corresponding to different pharmacies) may further be utilized to generate a list of potential pharmacies to include in the offers (e.g., the pharmacies with the lowest MAC list prices for the prescription drug may be selected).

It should be understood that for prescription drugs for which multiple different generic versions are available, different pharmacies may stock different ones of these generic versions. The APBM may identify or infer which particular generic version a particular pharmacy stocks in its inventory based on claims data that it may receive from a claims processor. In other embodiments in which the APBM has direct relationships with pharmacies, the APBM may receive an indication directly from the pharmacy as to which generic version of a particular drug it stocks. In one embodiment, the APBM may store an indication of which generic drug a particular pharmacy stocks and retrieve this data for use in either selecting pharmacies within the search area (e.g., if the APBM is searching for pharmacies that stock a particular generic drug), such as in pharmacy data 214 of memory 202, FIG. 2).

In many cases, multiple locations for a given pharmacy may be identified within the search area. Accordingly, in block 328, if multiple pharmacy locations (i.e., pharmacy retail stores) are identified for a given pharmacy, the system selects a subset of these (e.g., one) to include in the plurality of offers that are being assembled for the current prescription. For example, in accordance with one embodiment the pharmacy location that is closest to the search location (e.g., based on linear distance) may be selected for each pharmacy.

In accordance with some embodiments, the offers that are assembled and output to a consumer are generated and organized such that in the plurality of offers there is at least one offer that includes a pharmacy location in each of a plurality of zones (e.g., distance zones) relative to the search location. In accordance with some embodiments, three (3) zones may be utilized, as follows: (i) a first zone (which may, in accordance with some embodiments, be referred to as a “comfort zone”) which is defined by the shortest relative distance to the search location; (ii) a second zone (which may, in accordance with some embodiments, be referred to as an “economy zone” and is farther from the search location; and (iii) a third zone (which may, in accordance with some embodiments, be referred to as a “savers zone”) and be the furthest from the search location.

In accordance with some embodiments, an economy zone may be defined as being wide enough to capture the three (3) closest pharmacies of the search location, an economy zone may be defined as the next five (5) closest pharmacies of the search location and a savers zone may be defined as being all other pharmacies of the search location. In other embodiments, such zones may be defined via other parameters (e.g., distance from the search location, such that the first zone is within X miles of the search location, the second zone is located between X and Y miles of the search location and the third zone is located between Y and Z miles of the search location, where Z>Y>X).

While such zones may be utilized internally by the system to generate offers for a consumer, the indication of which offer corresponds to which zone may not be explicitly indicated to the consumer (e.g., the distance of a particular pharmacy location may be indicated in an offer, but not that it is in a “economy zone” as such).

A zone categorization of a particular pharmacy location may be utilized by the system to define other parameters of an offer in which the pharmacy location is included. For example, in one embodiment the zone categorization of the pharmacy location may be utilized to determine a reward amount to be included in the offer that defines that pharmacy location (e.g., by determining what percentage or portion of the difference between the PBM Price and the Pharmacy Reimbursement Amount corresponding to that offer should be included as a reward defined by the offer). In accordance with some embodiments, the APBM system may encourage a consumer to fill a prescription at a pharmacy location that maximizes savings for the consumer and/or the consumer's PBPP by presenting the consumer with an offer that defines a pharmacy with a relatively low Patient Price (based on a relatively low Pharmacy Reimbursement Amount) even though the pharmacy location is not located at a convenient distance from the consumer's location.

In accordance with some embodiments, if a pharmacy location that is within the search area for a prescription has a very low Patient Price (or has a relatively large differential between the PBM Price and the Pharmacy Reimbursement Price) but is within relatively further distance from the search location (e.g., within the economy zone or within the savers zone), the offer may include a relatively larger reward amount in order to encourage the consumer to accept the offer and fill the prescription at an inconvenient location but one that results in relatively higher savings (vis-à-vis a PBM Price) to the consumer and/or the consumer's PBPP. In another embodiment, a use of the zone categorization may be to allow the system to ensure that the plurality of offers that is ultimately output to the consumer includes at least one pharmacy location from each zone.

It should be understood that the number of zones (and the distance from the search location that each zone is defined by) may differ based on preference of the APBM and/or the PBPP and the embodiments described herein are not dependent on any particular number of zones or any particular distances or other parameters that defines each zone. Once a list of pharmacy locations is determined via sub-routine 300B (and, in some embodiments, a zone categorization is assigned to each such location), the offer assembly process may return to process 300A.

Returning once again to process 300A (FIG. 3A) and block 318 thereof, once a list of pharmacy locations is determined (e.g., via a sub-routine such as sub-routine 300B of FIG. 3B), the system continues to further assemble the plurality of prescription fulfillment offers by determining a Consumer Price for each pharmacy location. In accordance with some embodiments, determining the consumer price may also comprise determining a penalty or reward (if any) to be included in a particular offer. An example process 300C will now be described, with reference to FIG. 3C, to illustrate one method for how a Consumer Price may be calculated for a particular offer (including a determination of whether the offer should include a penalty or a reward, as described below).

Turning now to FIG. 3C, the process 300C illustrated therein is an example sub-routine that may be executed for each offer being assembled (e.g., for each pharmacy that will be defined in a particular offer), such that a respective Consumer Price to be included in each offer is determined. In accordance with some embodiments, process 300C may be performed by Pricing Module 224 (FIG. 2). It is again noted that process 300C is performed to determine a Consumer Price in scenarios where the consumer is no longer within the deductible period of his/her pharmacy benefit plan or the prescription drug for which offers are being assembled is a deductible exempt drug (such that the PBPP would be responsible for paying the PBM Price to the PBM if the PBM were to be utilized for filling the prescription).

At block 342 of process 300C, a Baseline Price is determined for the first offer being assembled. In accordance with some embodiments, the Baseline Price may be determined to be either a PBM Price or a Standard Baseline Price. In accordance with some embodiments, if the consumer is utilizing the APBM as an alternate prescription fulfillment mechanism parallel to the traditional PBM channel corresponding to the consumer's pharmacy benefit plan (the PBM associated with the consumer's PBPP), the Baseline Price may be determined to be the PBM Price for the prescription drug. As described herein, the PBM Price is the price the PBM would charge the PBPP for facilitating a fulfillment of a prescription of the drug under the consumer's pharmacy benefit plan. In accordance with one embodiment, the APBM may receive and store the PBM Prices for various PBMs from one or more sources and store them (e.g., in the drug pricing data 220 and/or the PBPP data 212 of memory 202, FIG. 2). For example, in one embodiment each PBPP with which the APBM has a relationship may provide the ABPM with a schedule of PBM Prices that it is charged by the PBM managing its pharmacy benefit plans. Thus, in one embodiment, if it is determined that a PBM Price is to be used for purposes of calculating a Consumer Price for an offer, the PBM Price may be retrieved from a database of the APBM (e.g., drug pricing data 220 of memory 202, FIG. 2).

In accordance with some embodiments, the APBM may not have direct access to, or confirmation of, a PBM price for a particular prescription drug. In such scenarios, the APBM may be operable to determine an estimated PBM Price, which may comprise a PBM Price that includes one of the following discount factors: (i) a generic discount factor (e.g., (0.39)*AWP of a generic drug); and (ii) a brand discount factor (e.g., (0.85)*AWP of the brand drug. The AWP comprises the “average wholesale price” for the drug, which is a published “sticker” price or average price for which wholesalers are said to sell the drug but that is not typically a true price that any entity pays for the drug (e.g., a MAC list price may be based on the AWP, and the Drug Cost a pharmacy paid to a wholesaler for the drug may be a portion of the AWP).

In accordance with some embodiments, the brand discount factor may comprise an estimated rebate amount negotiated between a PBM and a drug manufacturer (which, in some circumstances, is at least partially passed on to a PPBP by the PBM) may be determined based on how many acceptable product substitutes (e.g., generic versions or brands that are similarly effective for a given condition) there are available for the brand drug (the more brand or generic substitutes there being available, the higher the brand discount factor). The following table illustrates one brand discount scheme that may be applied based on the number of product substitutes available for a brand drug:

TABLE 1
Brand Discount Factors

| # of drug alternatives | Estimated Rebate Amount |
|---|---|
| 6+ | 60% |
| 3-5 | 40% |
| 1-2 | 20% |
| 0 | 10% |

In accordance with one embodiment, the number of drug alternatives may be derived using online published exclusion lists from third party services such as Express Scripts™ or Caremark™ For example, if the exclusion list of Express Scripts™ specifies that drug A is excluded and drug B, C and D can be used instead, we would assume that each of drug A, B, C and D have 3 substitutes and assign each of them an estimated rebate of 40% based on the table above. In one embodiment, data such as that illustrated in Table 1 may be updated (e.g., periodically) based on new estimates of rebate levels from various sources. In accordance with one embodiment, the determination of the number of acceptable substitutes may be determined by polling clinical experts to get their views on which drugs can be substitutes for other drugs. In one embodiment, the APBM may “crowd-source” this information and allow qualified medical practitioners to weigh in with their judgments on substitutability and make such information available to consumers (e.g., by informing consumers and/or PBPPs that “X % of medical practitioners believe ‘A’ can be substituted for ‘B’”).

In another embodiment (e.g., in an embodiment in which the consumer and/or the PBPP is using the APBM as its stand-alone prescription fulfillment mechanism, not as a mechanism running in parallel with a PBM prescription fulfillment option), the Baseline Price may be determined to be a Standard Baseline Price calculated in accordance with a formula. For example, in one embodiment the Standard Baseline Price may be determined to be the lowest of:
(i) the minimum Pharmacy Reimbursement Amount of the Pharmacy Reimbursement Amounts corresponding to a plurality of pharmacies identified as being within a first zone (e.g., pharmacies identified in accordance with process 300B to be within a comfort zone of the consumer's home location for the current prescription);
(ii) 120% of the lowest Pharmacy Reimbursement Amount of the Pharmacy Reimbursement Amounts corresponding to a plurality of pharmacies identified as being within a second zone (e.g., pharmacies identified in accordance with process 300B to be within an economy zone of the consumer's home location for the current prescription); and
(iii) 100% of the third lowest Pharmacy Reimbursement Amount of the Pharmacy Reimbursement Amounts corresponding to a plurality of pharmacies identified as being within the second zone (e.g., pharmacies identified in accordance with process 300B to be within an economy zone of the consumer's home location for the current prescription).

It should be understood that the above example formula for determining a Standard Baseline Price is intended as a non-limiting example and the embodiments described herein are not dependent on any particular formula for calculating a Standard Baseline Price. For example, other calculations that are based on a consideration of the Pharmacy Reimbursement Amounts corresponding to the pharmacies determined in process 300B may be substituted.

Next, in block 344, the Pharmacy Reimbursement Amount is identified. This is a determination of the monetary value that the pharmacy for which an offer is being assembled is expecting to be reimbursed by the PBM corresponding to the consumer's pharmacy benefit plan, if the consumer were to fill the prescription using the PBM channel. In accordance with some embodiments, the Pharmacy Reimbursement Amount may comprise an estimated or inferred Pharmacy Reimbursement Amount that is estimated or inferred by the APBM based on available but indirect information (since the Pharmacy Reimbursement Amount is often a confidential monetary value defined in a contract between a pharmacy and a PBM, and pharmacies are often contractually obligated to maintain such information as confidential). In accordance with one embodiment, the APBM may estimate or infer a Pharmacy Reimbursement Amount for respective prescription drugs as corresponding to particular pharmacies based on claims history data it receives from a claims processor (a third party entity that handles processing of reimbursements of Pharmacy Reimbursement Amounts from PBMs to pharmacies for prescriptions filled by the pharmacies). For example, the APBM may be operable to determine claims history information from a claims processor to determine which generic version of a drug a given pharmacy is stocking in its inventory. In one embodiment, the APBM may have access to a real time feed to one or more claims processors (in other embodiments, the APBM may receive this data periodically or non-periodically). The APBM may be operable to identify the particular generic being stocked by a given pharmacy based on the NDCs in the claims and therefrom determine the AWP for that generic using the published AWP lists that are publicly available (“NDC” being a National Drug Code, which is a unique 10-digit, 3-segment number that serves as a universal and unique product identifier for human drugs in the United States). In accordance with one embodiment, the APBM may then be operable to further estimate the Pharmacy Reimbursement Amount based on the AWP or MAC list corresponding to the particular brand or generic stocked by that pharmacy using published AWP or MAC lists and thus infer or estimate the Pharmacy Reimbursement Amount for a particular drug if it is filled at a particular pharmacy. It should be noted that the order of steps 342 and 344 may be reversed or the determinations of each may be performed simultaneously.

In accordance with some embodiments, the APBM may store an indication of various pricing contracts, each corresponding to a different PCN identifier, which list actual or estimated Pharmacy Reimbursement Amounts for specific prescription drugs. For example, in one embodiment the drug pricing data 220 (FIG. 2) may store an indication of the PCN identifier corresponding to each available Pharmacy Reimbursement Amount. In accordance with some embodiments, determining one or more Pharmacy Reimbursement Amounts for purposes of assembling one or more offers may comprise determining which pricing contract offers the lowest price (or the lowest set of possible prices) for the prescription drug for which offers are being assembled and identifying the PCN identifier corresponding to each such Pharmacy Reimbursement Amount that is selected for use in assembling an offer (for purposes of including it in a dynamically generated electronic prescription card should the offer be accepted, as described in more detail herein and particularly with reference to FIG. 5).

In block 346, a reward or penalty value to be included in an offer is determined. In one embodiment, the functionality of determining a reward or penalty may be performed by the pricing module 224 as part of determining the Consumer Price while in other embodiments it may be performed as a separate sub-routine or by a different software module. This comprises comparing the Baseline Price (whether using the PBM Price as the Baseline Price or the Standard Baseline Price as the Baseline Price) to the Pharmacy Reimbursement Amount. If the Baseline Price is higher than the Pharmacy Reimbursement Amount, then it is determined that a potential reward should be calculated for inclusion in the current offer being assembled. If, on the other hand, the Drug Cost is higher than the Baseline Price, then it is determined that a penalty should be included in the Patient Price of the offer being assembled. Either the penalty value or the reward value may be based on the difference between the Baseline Price and the Drug Cost, although neither needs to be equal to that difference. In one embodiment in which a penalty is determined to be appropriate, the entire difference between the Drug Cost and the Baseline Price is determined to be the penalty and the penalty is added to the co-pay (or co-insurance amount, if any) that the consumer is responsible for paying under the terms of his pharmacy benefit plan and the sum is determined to be the Consumer Price for the current offer.

In accordance with some embodiments, if the Pharmacy Reimbursement Amount is less than PBM Price for a given pharmacy/offer, then the system determines that a Reward should be offered to the consumer for filling the prescription at this pharmacy. This is because having the consumer fill the prescription at the pharmacy will only cost the PBPP the lower Pharmacy Reimbursement Amount rather than the higher PBM Price. The value of the Reward may be based on the difference between the PBM Price and the Pharmacy Reimbursement Amount. In one embodiment, the system may be programmed to provide to the consumer as a Reward a certain percentage of the difference between the PBM Price and the Pharmacy Reimbursement Amount (e.g., 50%). In some embodiments, the percentage or portion of this difference that is to be offered to the consumer as a Reward may be selected by the PBPP of the consumer's pharmacy benefit plan (e.g., some PBPPs may desire for the consumer to share in more of the cost savings that will result from the consumer filling the prescription at this pharmacy via the APBM system than will others). Thus, in such latter embodiments, the APBM may be programmed to retrieve the percentage or portion as selected by the consumer's PBPP in order to calculate the final Reward value. Alternately, the calculation of the Reward to be included in an offer may be done in block 320 of process 300A (in the same or similar manner as described with respect to block 346 of process 300C).

If the Pharmacy Reimbursement Amount is determined to be higher than the PBM Price (such that it would cost the PBPP more if the consumer were to fill the prescription at this pharmacy via the APBM system rather than via the traditional PBM channel), a Penalty may be determined as appropriate for inclusion with the offer. The Penalty value may be based on (e.g., equal to) the difference between the Pharmacy Reimbursement Amount and the PBM Price. The Penalty value may be, for example, set such that the PBPP is made whole and does not incur any additional costs if the consumer accepts an offer for which the Pharmacy Reimbursement Amount is higher than the PBM Price (and/or to discourage the consumer from accepting such an offer).

Once the Reward value or the Penalty value are set, the Consumer Price is assigned for the offer in block 348 (the Consumer Price is also referred to a consumer's out-of-pocket cost or OPC herein). The Consumer Price may, in accordance with some embodiments, be set to be the consumer's co-pay amount (and co-insurance amount, if any) plus the penalty value (if any had been determined in block 346). In accordance with one embodiment, the consumer's co-pay (or co-insurance) amount may be retrieved from a record defining the consumer's pharmacy benefit plan (e.g., from consumer data 210, memory 202, FIG. 2). In accordance with some embodiments, the Consumer Price may not necessarily be set to the co-pay amount, even if the consumer has already satisfied the deductible amount and would otherwise be expected to pay the co-pay amount when filling the prescription via the traditional PBM channel. In accordance with some embodiments, the Consumer Price may be set to be the lower of the co-pay amount and the Pharmacy Reimbursement Amount.

The Reward value (if any, as determined in block 346) is also assigned to the offer. If there is an additional offer being assembled for the consumer (i.e., if the offer currently being assembled is not the last of the plurality of offers being assembled in response to a request from the consumer), the process 300C may return to block 342 and repeated for the pharmacy of the next offer. Otherwise, the sub-routine 300C may end and the APBM may return to block 318. If the Reward value had not been calculated as part of the calculation in block 346 of process 300C, it may be calculated in block 320 of process 300A.

Reference will now be made to FIGS. 4C-4F, which illustrate how a result of process 300A (including sub-routines 300B and 300C) may be output to a consumer via an APB interface. Turning first to FIG. 4C, illustrated therein is a screen 400C that may be output on a mobile device 400 once a plurality of offers have been assembled for a consumer (e.g., the consumer who requested the offers by inputting information via the screen 400A and confirming the prescription details in screen 400B). For purposes of an illustrative example only, it may be assumed that it was determined (based on the pharmacy benefit plan information of the consumer stored by the APBM) that the consumer is no longer within a deductible period of the plan (i.e., the consumer has met the deductible amount of the plan, such that going forward the consumer is only responsible for a predetermined co-pay amount for prescriptions). In accordance with some embodiments, three (3) pharmacy fulfillment offers have been assembled for the consumer and are being output in area 430 of screen 400C. The first offer, 430A, defines a first pharmacy (Walmart™) and a first pharmacy location of that pharmacy that is indicated by the distance and estimated travel time relative to the consumer's search location (indicated in area 426 as being the consumer's home address). In some embodiments, if the consumer were to select the location symbol or name of the pharmacy, the consumer may be presented with specific information about the pharmacy location (e.g., an address and/or directions, in a pop-up window). The offer 430A also defines a Consumer Price of $5 (as indicated in the “Your Price” column of area 430) and a reward of $30 to be provided to the consumer if the consumer were to accept this offer and select this pharmacy location for fulfillment of the prescription in accordance with the terms of the offer. The second offer, offer 430B, defines a second pharmacy Durham™ (at a specific location that is a specified distance and estimated travel time from the consumer's home location) and that has a Consumer Price of $6 and a Reward of $30. Finally, the third offer 430C defines a third pharmacy Qualitas™ (at a specific location that is a specified distance and estimated travel time from the consumer's home location) and that also has a Consumer Price of $6 and a Reward of $30. Thus, as a visual comparison of the three example offers reveals, the offer that will be most financially beneficial to the consumer (because it has the lowest Consumer Price but the same Reward as the other two) is the least convenient in terms of distance and travel time.

It should be noted that screen 400C includes a mechanism, in area 428, via which the consumer may toggle between a view of the Reward corresponding to each offer and a view of the Estimated Non-APBM Price corresponding to each offer. Screen 400D (FIG. 4D) illustrates the alternate view that can, in some embodiments, be output to the consumer if the consumer selected to view the Estimated Non-APBM Prices rather than the Rewards corresponding to each offer. As each of offers 430A, 430B and 430C indicate in area 430 of screen 400D, the Estimated Non-APBM Price for each offer is $10. In the present example, it may be assumed that the co-pay the consumer would be expected to pay if he/she were to fill the prescription using the traditional PBM channel (i.e., not using the APBM app/system), is $10. The Estimated Non-APBM Price is the price the consumer would pay for filling the prescription using the traditional PBM channel (in the present example this being the $10 co-pay). Thus, the consumer can readily appreciate that if he/she were to fill the prescription via the APBM system, he/she would pay $5 or $6 out-of-pocket and receive a $30 reward while if he/she were to not accept any of the offers in area 430 and instead choose to fill the prescription in the conventional manner using the PBM channel, his/her out-of-pocket cost would be $10 and he/she would receive no reward.

It may be assumed, for purposes of the example of FIGS. 4C-4F, that the Pharmacy Reimbursement Amounts for offers 430A, 430B and 430C were determined (e.g., via sub-routine 300C) to be $5, $5 and $6, respectively. Thus, in accordance with some embodiments, the Consumer Price was assigned in each offer to be the Pharmacy Reimbursement Amount, since in each case this was lower than the consumer's co-pay amount. Turning now to FIG. 4E, illustrated therein is a screen 400E that may be output to the consumer in response to the consumer selecting one of the offers output in screen 400D. For purposes of the present illustrative example, it may be assumed that the consumer selected offer 430A and screen 400E has popped up (as window 435A) to indicate the details of the offer (including the details of the prescription) and provide the consumer with an opportunity to accept the offer (e.g., by actuating the virtual button 434 on screen 400E). It should be noted that the screen 400E outputs additional detail regarding the offer 430A, including a breakdown of (i) the employee (consumer) OPC (which is $5 in the present example), 431; (ii) the employer (PBPP) cost (which is $0, since the $5 to be paid by the consumer/employee is the Pharmacy Reimbursement Amount in this case, such that the employer is not responsible for any additional payment; and (iii) the Reward to be provided to the consumer upon fulfillment of the prescription in accordance with the offer (which is $30 in the present example), 433.

It may be assumed, for purposes of the present example, that the PBM Price of the prescription drug that is the subject of offers 430A, 430B and 430C is $65. In other words, if the consumer were to fill the prescription via the traditional PBM channel and not via the APBM system/app, the PBM would charge the PBPP $65 for facilitating the fulfillment of this prescription and that the consumer would be responsible for $10 of this PBM Price (the consumer's co-pay amount) while the PBPP (e.g., employer) would be responsible for the remaining $65. Thus, if the consumer were to choose to fill the prescription via the APBM system/app and pay only the $5 or $6 Consumer Price (depending on which offer the consumer accepts), and the Consumer Price being the Pharmacy Reimbursement Amount, the PBPP would not be responsible for paying its portion of the $65 PBM Price to the PBM. The difference between the PBM Price and the Pharmacy Reimbursement Amount is $60 (for the pharmacy with the $5 Pharmacy Reimbursement Amount) or $59 (for the pharmacy with the $6 Pharmacy Reimbursement Amount). In accordance with some embodiments, the Reward for each offer may be set to be 50% of this difference, rounded to the nearest dollar (of course, any % or portion may be used and the 50% is used merely as a non-limiting example). This calculation accounts for the $30 reward to the consumer, which the PBPP will ultimately fund because paying this $30 Reward to the consumer still results in significant savings to the PBPP.

In accordance with some embodiments, if a consumer elects to accept an offer, the consumer's acceptance may be stored in the system and an electronic prescription card may be generated by the system. An example of such a prescription card for offer 430A is illustrated in area 435B of screen 400F (FIG. 4F). The electronic prescription card may be utilized by the consumer to fill the prescription at the pharmacy. Consistent with at least some embodiments, the electronic prescription card 435B may include details of the prescription (in area 435B-1) and APBM details that will allow a pharmacy claims system to process a claim for the prescription (in area 435B-2). With respect to area 436B-2 and the prescription BIN and prescription PCN identifiers illustrated thereon, it should be noted that the digital prescription card may include a BIN (Bank Identification Number, which identifies a PBM (or the APBM, in the case of embodiments described herein, associated with the processing of the prescription), a PCN (Processor Control Number, which identifies the particular APBM or PBM Network or pricing contract corresponding to the prescription) and a Group identifier (which identifies a plan of the consumer attempting to fill the prescription). Each of these identifiers helps facilitate a pharmacy and/or claims processor in processing the prescription by indicating the pricing contract that corresponds to the prescription fulfillment offer.

As described in more detail with respect to FIG. 5 and process 500, one novel aspect and result of the APBM processes and systems described herein is that a BIN/PCN identifier combination is dynamically generated for each accepted offer that is processed by the APBM, which BIN/PCN identifier combination is included in the electronic prescription card that is generated in response to the consumer accepting an offer. The BIN identifier identifies the APBM as being associated with the prescription (as opposed to a traditional BPM) and the PCN identifies the particular pricing contract that was used to assemble the offer and calculate the Consumer Price defined by the offer. The resulting BIN/PCN identifier combination that is dynamically determined for each offer and for each electronic prescription card that is uniquely generated for each accepted offer allows the consumer to obtain the prescription drug at the Consumer Price defined by the offer (such that there are no surprises or disappointments at the point-of-sale, such as sometimes happens when a consumer attempts to use a conventional drug “discount card” or “coupon” when filling a prescription and it is refused or not honored by the pharmacy). In accordance with some embodiments, the electronic prescription card that is dynamically generated and includes a BIN/PCN identifier specific to the transaction or accepted offer provides the consumer and pharmacy certainty as to what price will be paid by the consumer and what amount will be reimbursed to the pharmacy for this prescription. For example, in the example of FIG. 4H the PCN identifier is listed as “FLIPT1”, indicating the particular price list or PCN price network that corresponds to the prescription fulfillment offer.

Returning now to block 312, in this block a Consumer Price may be calculated for each of a plurality of offers being assembled for a consumer/prescription in a scenario in which the consumer has not yet met his deductible amount and the prescription drug for which prescription fulfillment offers are being assembled is not a deductible exempt drug. In other words, the consumer is responsible for the full cost of the prescription drug, not just the co-pay (whether this be the PBM Price if the consumer chooses to fill the prescription using the traditional PBM channel or the APBM Consumer Price if the consumer chooses to fill the prescription using the APBM system/app). In such a scenario, no rewards are calculated or provided to the consumer (since the PBPP is not responsible for any portion of the cost of the prescription drug and thus does not have any potential savings to pass on to the consumer in the form of a reward). Rather, the Consumer Price is determined to be the Pharmacy Reimbursement Amount for each pharmacy defined by each respective offer. In some embodiments, the Consumer Price may also include a Penalty that is added to the Pharmacy Reimbursement Amount. The Penalty may be added if, as described with respect to block 346 of sub-routine 300C, the Pharmacy Reimbursement Amount is determined to be higher than the PBM Price or estimated PBM Price for the offer. In block 314, the PBM Price or estimated PBM Price is determined. The determination of a PBM Price or estimated PBM Price was described with respect to block 342 of process 300C and will not be repeated herein for purposes of brevity.

In block 316, a menu of the plurality of offers assembled for the consumer is output via an APBM GUI. If the process 300A flows to block 316 from block 320, the output comprise information such as indicated in FIGS. 4A-4F. If, on the other hand, the process 300A flows to block 316 from block 314 (in which scenario there are no Rewards to offer to the consumer), the output may include, for each offer included in the menu of offers, a comparison of the Consumer Price (which often will be based on the Pharmacy Reimbursement Amount for each pharmacy) to the PBM Price or estimated PBM Price that the consumer would otherwise pay for the prescription, thus allowing the consumer to appreciate the savings he/she can realize (which can be considered a reward in and of itself) by filling the offer via the APBM System and thus paying the Pharmacy Fulfillment Price rather than the typically higher PBM Price.

In accordance with some embodiments, once a plurality of offers is output to a consumer and the consumer accepts an offer, additional steps or another sub-routine of APBM may be initiated. For example, as described herein, in some embodiments the consumer pays the Consumer Price for an accepted offer directly to the APBM using the APBM app and thus has nothing to pay to the pharmacy at the point-of-sale when filling his/her prescription using the APBM electronic prescription card. In such embodiments, once the consumer accepts an offer he/she may be routed to a payment process via which the consumer provides payment of the Consumer Price to the APBM. In one embodiment, the consumer may previously have stored credit card or other financial account information with the APBM (or provided with the APBM to charge fees to such credit card or other financial account) and thus the payment process may simply comprise having the consumer confirm the Consumer Price that is to be charged and authorizing the payment. In other embodiments, the consumer may need to input payment information. In yet other embodiments, the consumer may need to provide the Consumer Price at the point-of-sale to the pharmacy and the pharmacy and APBM may reconcile at a later point.

Further, once a consumer accepts an offer the APBM may transmit information regarding the offer to a claim processor (e.g., claim processor 150) that will be expected to process the claim from the pharmacy location defined by the accepted offer. The information transmitted to the claim processor may be whatever information the claim processor requires to approve and process the claim when a request for it comes through from the pharmacy location. In accordance with some embodiments, the information may comprise the information included on the electronic prescription card generated for the accepted offer (as described in more detail with respect to process 500, FIG. 5). Thus, the claim processor may, in accordance with some embodiments, open a pending claim or record for the offer (which, in some embodiments, may expire after a specified time) upon receiving the information from the APBM.

Additionally, once a consumer accepts an offer a dynamically generated electronic prescription card that includes the BIN/PCN identifier combination that is specific to the accepted offer (wherein the PCN identifies the pricing network or contract based on which the Consumer Price defined in the offer was generated and which allows the Consumer Price to be guaranteed to the consumer by the APBM). A prescription card generation process, such as the example one described with respect to FIG. 5, may thus be initiated upon a consumer accepting an offer.

Turning now to FIG. 4G, illustrated therein is an example screen 400G, which may be output to a consumer who is a registered user of the APBM, and allow the user to view and manage the various prescription offers the consumer has accepted and/or filled via the APBM. In accordance with some embodiments, area 441 of screen 400G allows a consumer to view or manage (e.g., delete) information on prescriptions which the consumer has entered information for but for which the consumer has not yet selected a prescription fulfillment offer (in accordance with one embodiment, the user may request offers to be assembled for such a prescription by actuating the virtual button 442). Also in accordance with some embodiments, area 443 allows the consumer to view or access information on prescriptions for which the user has accepted a fulfillment offer but which prescription the consumer has not yet filled (in accordance with some embodiments, such an offer may expire if not filled within a predetermined period of time, as indicated in area 443). Also in accordance with some embodiments, area 444 allows the consumer to view or access information on prescriptions that the consumer has filled via the APBM system.

It should be noted that a consumer may be able to view and manage prescriptions for other persons (e.g., other family members who are covered under the consumer's pharmacy benefit plan). Thus, in such embodiments, a screen such as screen 400G may allow the consumer to view and manage prescriptions for such other people (e.g., view an electronic prescription card for a child or spouse's prescription such that the consumer can go to a pharmacy to fill the prescription).

Turning now to FIG. 4H, illustrated therein is an example screen 400H that allows a consumer to view or manage (e.g., redeem) rewards earned by the consumer by accepting prescription fulfillment offers via the APBM. In accordance with some embodiments, the storing, managing and facilitating of pending and earned rewards, as well as the redemption of earned rewards, may be facilitated by reward module 228 (FIG. 2).

In accordance with some embodiments, some offers output to a consumer may include an indication of a Reward to be provided to the consumer upon the consumer accepting the offer and filling the prescription in accordance with the terms of the accepted offer. In some embodiments, such Rewards may define monetary value amounts that can be redeemed at partner merchants (e.g., Amazon™ or Walmart™). For example, in some embodiments such Rewards may be used as store credit or to purchase gift cards to such partner merchants. Area 451 of the example screen 400H indicates a value of Rewards that are pending. Rewards that are pending may comprise, for example, Rewards for offer(s) the consumer (or others associated with the consumer's APBM account, such as children or a spouse that may be covered under the consumer's pharmacy benefit plan) has accepted but has not yet filled the prescription in accordance with the terms of the offer(s). Area 452, on the other hand, indicates a value of Rewards that are available for redemption (e.g., Rewards for offers that have been accepted and for which the prescriptions have been filled in accordance with the terms of the offer). In accordance with some embodiments, redeeming a value of Rewards may comprise (i) using the Rewards to purchase gift cards or store credit for use with one or more merchants; (ii) requesting a check or deposit of the monetary value to be made to a bank account associated with the consumer; or (iii) requesting that a value of the Reward be applied to a Consumer Price corresponding to a new offer output to the consumer via the APBM.

Referring now to FIG. 5, illustrated therein is a process 500 that may be performed by an APBM in accordance with some embodiments described herein. Process 500 comprises an example process for dynamically generating an electronic prescription card, on a per transaction basis, responsive to an offer being accepted by a consumer.

As a preliminary matter, before describing the details of the process 500, an overview of a traditional process (i.e., one involving a PBM and prior to the existence of the APBM systems and processes described herein) for how a claim processor treats pharmacy transactions is described. In accordance with prior art systems and methods, a consumer selects a pharmacy benefit plan (and thus the corresponding formulary) and is issued a physical prescription plan card which indicates the BIN/PCN identifier of the PBM corresponding to the plan (among other identifiers, such as a Group and Member identifier). When a consumer is provided with a prescription by a medical professional, he/she takes it to a pharmacy (or it is called in to the pharmacy by the medical professional). The pharmacy runs the prescription that arrives from the doctor (digital or physical script) and enters in the prescription plan card details (most of the time these are keyed in once and then stored in the pharmacy system). The pharmacy system then uses the prescription card information to route the prescription details to a claim processor. The claim processor then (i) checks eligibility by confirming if the individual who's name is on the prescription is still active in the census file that comes from the PBPP associated with the plan identified via the prescription plan card; (ii) cross checks the prescription details against the plan formulary to determine if the drug is covered and/or if any restrictions apply, and (iii) adjudicates the claim by identifying which plan the consumer is on and checking whether the consumer has met any applicable deductibles. The claim processor then sends back a message to the pharmacy confirming the consumer is eligible, the drug is covered, and indicating the amount the pharmacy should charge the consumer at the point of sale, such as the co-pay amount if the consumer has satisfied the deductible amount or the PBM Price if the consumer has not yet satisfied the deductible amount. If either the eligibility or drug coverage are not confirmed by the claim processor, the message that is sent to the pharmacy from the claim processor indicates that the prescription is “rejected” (and may include a brief description indicating the reason for the rejection).

As described above, a PBM enters into PPAs with various pharmacies, a PPA between a PBM and a pharmacy defining the rates at which a pharmacy company will be reimbursed by the PBM for both brand and generic drugs. The PPAs are often confidential and include the pricing terms between a PBM and the pharmacy, one of which terms may specify an amount of money the PBM has to reimburse the pharmacy for drugs (usually structured as a Generic Effective Rate, GER, for generic drugs, which measures the reimbursement for an entire basket of drugs rather than pricing each drug individually; for example, a PPA may specify that for every $1 of “AWP” the pharmacy will receive $0.20 in reimbursement). A PBM then has resells their network of pharmacy agreements to a PBPP at a markup (e.g., an agreement with a PBPP may specify that the PBPP will only pay $0.30 on every dollar of AWP when the PBM has agree to pay pharmacies $0.20 on every dollar of AWP), such that the PBM profits from the spread between the pricing it reimburses to the pharmacy and the amounts it charges to its client PBPPs (e.g., in the present illustrative example the profit is a 50% mark-up, ($0.30-$0.20)/$0.20). However, to know exactly what price each drug needs to be reimbursed at, the PBM provides a MAC list to the pharmacy (and a separate one that's marked-up to the client PBPP) that essentially offers an itemized price list per drug. The individual prices on the MAC list, multiplied by the actual volumes filled for each generic drug, should come close to the target GER that the PBM contracted with the pharmacy. The PPAs includes a specific BIN and PCN identifier. The BIN identifies the PBM. And within that PBMs network, the PCN identifies a specific account that can have its own specific set of prices and unique MAC lists. Thus, a claim processor that receives a pharmacy fulfillment request from a pharmacy determines the BIN/PCN identifiers corresponding to the request (e.g., from the consumer's Prescription Plan card) and, if the request is approved based on the eligibility and drug coverage verifications, determines the price that the consumer is to be charged for the prescription based on the BIN/PCN and returns this information to the pharmacy. It should be noted that, in the traditional pharmacy fulfillment process involving a PBM and the pharmacy's communication with a claim processor when the consumer is filling a prescription, the consumer is using a static BIN/PCN set of identifiers that are on his/her prescription plan card issued by his/her PBPP and that identifies the PBM that the PBPP has contracted with and the particular PCN corresponding to that PBM contract/network.

Returning now to FIG. 5 and process 500 that illustrates an example process for dynamically generating an electronic prescription card, it should be noted that process 500 may be performed by prescription card module 232 (FIG. 2) and initiated upon a consumer accepting an offer for fulfillment of a prescription. In accordance with embodiments described herein, consumers using the APBM system for obtaining a guaranteed Consumer Price for a prescription drug are provided with a dynamically generated electronic prescription card to use when filling the prescription at the pharmacy. Thus, in accordance with embodiments described herein, a particular consumer filling prescriptions at a particular pharmacy location may be provided with a first electronic prescription card with a first BIN/PCN combination to use when filling a first prescription and a second electronic card with a second (and different) BIN/PCN identifier combination to use when filling a second prescription. As the same APBM corresponds to both prescriptions (identified in the BIN), this is different than if the consumer had utilized the traditional PBM process for filling the prescription because in that case the consumer would have utilized a static BIN/PCN combination (as defined on the prescription card issued from his/her pharmacy benefit plan) for both prescriptions since the PBM only utilizes a single and particular PCN for a particular PBPP (the one associated with the consumer's pharmacy benefit plan that is managed by the PBM).

In block 502 the consumer information to be included on the electronic prescription card is determined and added to the information to be included on the card (e.g., the name of the person named on the prescription, the Group identifier and Member identifier corresponding to the consumer, etc.). Such information may be retrieved, for example, based on consumer data 210 (FIG. 2) corresponding to the consumer who is logged into the system and for whom the offers were assembled and/or data input by the consumer when requesting the offers (e.g., via GUI screen such as that illustrated in FIG. 4A and/or 4B).

In block 504, the prescription details are determined and added to the electronic prescription card (e.g., the specific drug, form, dosage, and supply amount). This information may be based on information input by the consumer in a GUI screen such as that illustrated in FIG. 4A and/or 4B and information retrieved by the APBM when assembling the offers, such as during the drug selection process performed by module 222).

In block 506, the PCN identifier corresponding to the accepted offer is determined. For example, the PCN identifier corresponding to the particular Pharmacy Reimbursement Amount used to calculate the Consumer Price (e.g., as this determination may have been performed by the pricing module 222 and/or pharmacy selection module 224 in any of processes 300A-300C) may be determined and the PCN identifier corresponding to the price list/network from which the Pharmacy Reimbursement Amount was determined may be retrieved for inclusion on the electronic prescription card.

In block 508, the electronic prescription card that is transaction-specific in the sense that the information on it (including the particular BIN/PCN identifier combination) is only valid for filling the prescription defined in the accepted offer is generated and output to the consumer. The card is also stored on the APBM app such that the consumer can pull it up and show it at the pharmacy when filling the prescription.

As described herein, each electronic prescription card is generated responsive to an accepted offer and includes a BIN/PCN identifier combination that is specific to the corresponding accepted offer, since the PCN identifier is selected for inclusion on the card based on the Pharmacy Reimbursement Amount that was selected and used to calculate the Consumer Price for the accepted offer. Thus, unlike in the traditional PBM model in which a consumer is provided with a static prescription plan card to show at a pharmacy when filling a prescription (or the static information on which is stored at the pharmacy at which the consumer typically fills his/her prescriptions), consumers who utilize the APBM system to fill their prescriptions do not have static prescription plan cards because the BIN/PCN identifier combination is not static in the sense that it is not the same for all transactions facilitated by the APBM. The BIN/PCN identifier combinations for ABPM-facilitated pharmacy fulfillment transactions change because the app identifies and sources the best prices based on the pharmacy location and the drug the consumer is searching for. As described herein, the APBM has the ability to go through multiple pricing contracts (each corresponding to a different PCN) in order to determine where the best price will be pulled from, and each contract will have its own unique BIN/PCN combination. Thus, the APBM system generates a unique electronic prescription card for each pharmacy fulfillment offer.

It should be noted that an electronic prescription card should not be confused with the actual prescription script that is issued by a medical professional (whether it is written on a piece of paper by the medical professional, called in to the pharmacy directly or entered electronically into an online script system that the pharmacy can access). The electronic prescription card described herein as being generated by the APBM is more akin to the physical pharmacy, prescription plan card that is issued by the PRPP to the consumer, that the consumer presents at a pharmacy to show he/she has a pharmacy benefit plan to be used in processing payment for the prescription (e.g., the prescription card is used by the pharmacy to determine the consumer's co-pay). The actual prescription process (doctor prescribes/pharmacy receives the prescription from the doctor) remains unchanged by the APBM systems and methods. For example, when using the APBM system the consumer copies the prescription details as per his/her doctor's prescription into the APBM app to generate an electronic prescription card (based on the prescription fulfillment offer the consumer accepts from the APBM), to be used to facilitate the consumer's payment for the prescription (and the pharmacy location's reimbursement for filling the prescription). When using the APBM system to pay for a prescription, the consumer communicates to the doctor where he wants the prescription to be sent and then goes to that pharmacy (based on the offer the consumer accepted via the APBM). At the pharmacy, the consumer will need to show the pharmacist their electronic prescription card generated by the APBM (which might have different BIN/PCN identifier set than one that the consumer may have previously used when filling an APBM-facilitated prescription at this same pharmacy, as described herein). The pharmacy will key in the doctor's prescription and the details from the consumer's electronic prescription card (rather than the physical prescription plan card that a consumer may have from their PBPP and which conventional prescription plan card has a static BIN/PCN combination) into their pharmacy management system which then uses the Bin/PCN to route the claim to a specific processor (e.g., ScriptClaim™).

In accordance with some embodiments, the claim processor (e.g., claim processor 150) will typically already have a pending pre-approval from the APBM for a consumer/accepted offer at the time it receives this information from a pharmacy. Thus, in accordance with some embodiments, there may be no need for the claim processor to verify eligibility or formulary coverage, and adjudicate or determine the consumer out-of-pocket. The APBM system will already have confirmed all of these details when assembling the offers for the consumer and generating the electronic prescription card, which it sends to the claim processor ahead of the consumer attempting to fill the prescription at the pharmacy location defined in the accepted offer, such that the claim processor needs only to match up the pre-approval from the APBM with the pharmacy claim request and issue an approval (or in cases where information does not match up, a rejection).

In accordance with some embodiments, the APBM may optionally set thresholds for how far off any of on the data in the pre-approved information provided to the claim processor can be from the information the claim processor receives from the pharmacy in a claim request without being rejected by the claim processor. For example, if the consumer's electronic prescription card specifies that it is for 30 tablets but the actual script from the doctor is for 60 tablets, the APBM tolerance thresholds may be set such that the claim processor may nevertheless be authorized to approve this claim request despite the iris-match in the data defining the amount of supply.

Turning now to FIG. 6, illustrated therein is an example process 600 which comprises a process via which a system (e.g., APBM System 200 of FIG. 1 and/or FIG. 2) may output offers for different healthcare service providers that a consumer can visit. Today, different healthcare service providers charge different rates for the same or similar type of service (e.g., a physical, an evaluation with an allergist, an evaluation with a gastroenterologist, etc.). For example, different providers or facilities may have different rates contracted with different healthcare plans In accordance with some embodiments, the APBM System 200 may be operable to determine the prices charged by various healthcare providers (e.g., within a given geographical area) for the same or similar type of service and utilize this information to output to the consumer different price options for a plurality of healthcare providers. For example, the system may receive an input from a user indicating a type of healthcare provider (e.g., medical specialty) or health condition (e.g., stomach problems vs. ongoing headaches) and select, based on one or more factors such as a consumer's geographic location, a plurality of healthcare providers and/or facilities that are appropriate for the user to visit, along with an out-of-pocket cost to the consumer for each option (e.g., based on a price the provider or facility charges for the type of service the consumer is looking for and/or based on a reimbursement price that the system would need to provide to the healthcare service provider if the consumer were to go to the healthcare service provider upon accepting an offer to do so from the system). This allows consumers to make cost effective decisions similar to how they may be able to choose pharmacies with the lowest cost for the prescription drug they need.

The ABPM System 200 may, in some embodiments, enter into its own proprietary contracts or rate schedules with healthcare providers or facilities. In other embodiments, the APBM System 200 may obtain access to information on rates that the healthcare providers or facilities have agreed to with different entities (e.g., PBPPs) and utilize this information to compare rates and generate offers to a consumer. For example, in one embodiment the APBM System 200 may store in its memory a database or other records of rate schedules or price lists for various types of healthcare services, per service provider or facility and corresponding to different geographical areas or address information. Such rate data may be referred to as Healthcare Provider Rate Data herein and may define, in some embodiments, the reimbursement price that the APBM system would be obligated to pay a corresponding healthcare service provider for sending a consumer to that provider.

In step 602, the system receives from the consumer a request for offers pertaining to healthcare service providers or facilities. In some embodiments, the consumer and/or consumer's physician may provide certain information to the system when making such a request. For example, the consumer may indicate an area of specialty, a suspected medical condition or area of the body that the healthcare provider or facility should be qualified to assess (e.g., heart specialist vs. orthopedist). In another example, the consumer may indicate any preferences with respect to the healthcare provider or facility (e.g., gender, years of experience, level or type of education, diversity of available services, hours of operation, etc.).

In step 604, the consumer's preferred location (e.g. vis-à-vis the current request) is determined. This may comprise retrieving a location stored in the consumer's record (e.g., a home or work address) or receiving the location from the consumer (e.g., the consumer may be on vacation and input the location of the vacation at which the healthcare provider is needed). The maximum distance the consumer prefers to travel from the preferred location may also be received from the consumer or retrieved (e.g., based on a preference previously stored in association with the consumer). In some embodiments, a default preferred maximum travel distance (e.g., 5 miles) may be used by the system. In some embodiments, different offers for different distances from the preferred location may be output to the consumer (e.g., the longer the travel distance the lower the price to the consumer).

In step 606, the consumer's healthcare policy data (e.g., information on which providers or facilities are considered to be “in network” for the consumer's healthcare policy, how close the consumer is to meeting his/her deductible, etc.) are retrieved. This step may be similar to what is described with respect to step 306 (FIG. 3A) but be focused on the terms of a healthcare benefit policy other than a pharmacy benefit policy (i.e., the consumer's health insurance plan terms based on the specific plan the consumer is a member of). Whether the consumer is still within the deductible period and the relevant co-pay for this type of healthcare provider or facility are examples of the types of information related to the healthcare policy that may be retrieved in step 606 and utilized to generate offers for the consumer. If the consumer does not have a healthcare policy, this step may be omitted.

In step 608, any available rewards for use in generating offers for the consumer are retrieved, for healthcare providers or facilities that are valid candidates for the current request healthcare service providers or facilities that are within an acceptable travel distance from the consumer's preferred location and satisfy any relevant terms of the consumer's healthcare policy). For example, participating healthcare service providers or facilities may provide to the APBM certain rewards or monetary incentives to be utilized to steer consumers to them. For example, a particular physician's office may be offering a special rate on a given month in order to attract new patients or new patients that meet specific criteria (e.g., not current patients of that physician's office, patients of a certain age or gender, etc.). These rewards, if any, and the Healthcare Provider Rate Data stored by the APBM is then utilized by the system to generate a plurality of offers for the consumer and output (in step 610) these offers to the consumer (e.g., via the APBM app). In this manner, a consumer may be able to efficiently and timely view different options (and the price or out-of-pocket cost the consumer would incur for each) for different healthcare providers or facilities needed by the consumer.

Turning now to FIG. 7, illustrated therein is an example process 700 comprising an example process via which a system (e.g., APBM System 200) may be operable to generate and output to a consumer at least one offer for an alternate treatment option (e.g., a diagnostic test or follow-up procedures or tests) that the consumer should discuss with his/her healthcare provider in association with a diagnosis or potential diagnosis the healthcare provider has indicates for the consumer. Patients (consumers herein) are diagnosed as having a certain medical condition or potentially having a certain medical condition and healthcare providers submit claims to a consumer's health benefit plan provider or PBPP using diagnosis codes (also referred to as diagnostic codes here); these codes are then used as the basis for follow on procedures that might need to be done. The ABPM System may, in some embodiments, be operable to determine and/or store standard, recommended or generally acceptable practices regarding follow-up treatments, tests or procedures corresponding to certain diagnosis codes and be programmed to offer alternatives to the consumer (e.g., alternatives for the consumer to discuss with his/her healthcare provider) if the consumer's healthcare provider recommends a treatment, test or procedure that differs from what the APBM System has stored as the recommended treatment, test or procedure. For example, assuming a doctor diagnoses a patient with postural-orthostatic-hypotension and writes a prescription for a chest x-ray, the APBM System may recommend alternatives since research shows that x-rays in this diagnosis almost never show any findings that would change the course of treatment the doctor was going to prescribe anyway. In some embodiments, the APBM System may additionally offer incentives to the consumer to decline the treatment, test or procedure initially recommended by the consumer's doctor (or at least express reservations about it to his/her doctor and request additional discussion as to the effectiveness or need for the recommended treatment, test or procedure), similar to how the APBM System may offer prescription drug substitution recommendations.

Turning now to process 700, in step 702 the system may determine at least one diagnostic code provided by a healthcare provider for a patient, indicating the healthcare provider's diagnosis or potential diagnosis of the consumer's medical condition. The diagnostic code may be received by the system, for example, by having the consumer and/or healthcare provider enter it into a user interface of the APBM app. In another embodiment, the APBM System may determine the diagnostic code from a claim submitted by the healthcare provider, responsive to a visit from the consumer, to the consumer's health benefit plan provider or other entity.

Based on this diagnostic code determined in step 702, the APBM retrieves at least one recommended or acceptable treatment option corresponding to the diagnostic code (step 704). For example, the APBM may retrieve such treatment options based on diagnostic codes and records in a database such as diagnostic code data 242 as stored in database 202 (FIG. 2). A treatment option may comprise a prescription for a medication, a follow-up procedure or test (e.g., x-ray, CAT scan, blood test, biopsy, etc.) or a follow-up medical regimen (e.g., visit to a chiropractor). In accordance with some embodiments, the APBM System may store (e.g., in a database of records which stores diagnostic codes and the corresponding acceptable treatment options and/or corresponding unacceptable treatment options) information indicating acceptable treatment options for a given diagnostic code. The APBM System may also retrieve the consumer's healthcare benefit plan or policy and determine the relevant terms thereof (step 706). This step may be similar to what is described with respect to step 306 (FIG. 3A) but be focused on the terms of a healthcare benefit policy other than a pharmacy benefit policy (i.e., the consumer's health insurance plan terms based on the specific plan the consumer is a member of). Whether the consumer is still within the deductible period and the relevant co-pay for the type of treatment option corresponding to the diagnostic code received in step 702 are examples of the types of information related to the possible treatment options that may be retrieved in step 706 and utilized to generate offers of alternate treatment options for the consumer. If the consumer does not have a healthcare benefit plan or policy, this step may be omitted.

In step 708, the APBM System may be operable to output (e.g., via an interface of the APBM app) one or more offers for alternate treatment options that are considered acceptable for the diagnosis indicated by the diagnostic code determined in step 702. In accordance with some embodiments, the offers may also include a cost comparison (e.g., of out-of-pocket costs to the consumer) for the various treatment options. For example, based on pricing information available to the APBM (e.g., pricing for the different treatment options from different healthcare facilities, healthcare providers and/or pharmacies, as appropriate based on the type of treatment option), as well as the terms of the consumer's healthcare benefit plan or policy, the APBM may be able to inform the consumer of how much it would cost him/her out-of-pocket to choose the option recommended by the consumer's healthcare provider vs. at least one alternate treatment option determine by the APBM System 200. As discussed herein, in one embodiment the offers are intended to be discussed by the consumer with the consumer's healthcare provider prior to being accepted by the consumer (and, in some embodiments, the healthcare provider would need to formally modify the prescription for the recommended treatment option if it is decided that an alternate treatment option suggested by the APBM System 200 is acceptable to the healthcare provider).

Other adaptations of the APBM system described with respect to pharmacy benefit plans may be made that take advantage of the APBM system's capabilities vis-à-vis healthcare expenditures in addition to expenditures on prescription drugs, healthcare provider or facility services or diagnostic tests or treatment plans. For example, similar to the systems and processes described herein with respect to pharmacy benefit adjudication, in some embodiments the APBM System 200 may be operable to streamline the adjudication process for other types of healthcare benefits (e.g., eligibility to visit a particular healthcare provider or facility based on the consumer's particular healthcare policy rules) that today is heavily burdened by administrative and manual processes. The APBM may be operable to auto-adjudicate and check eligibility on the front-end such that healthcare providers lower the risk of rejected claims (e.g., billing code rejections), which may in turn allow the APBM System transactions to get preferential rates/discounts. In another example, APBM System adjudication capabilities may allow the APBM System to partner with a number of different network providers and enhance its attractiveness such that it can negotiate good rates or deals for its users.

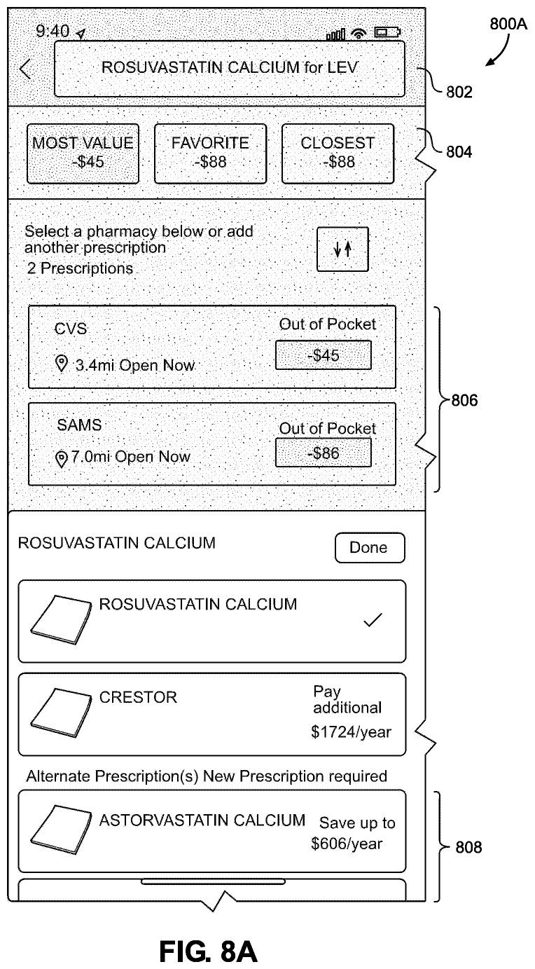

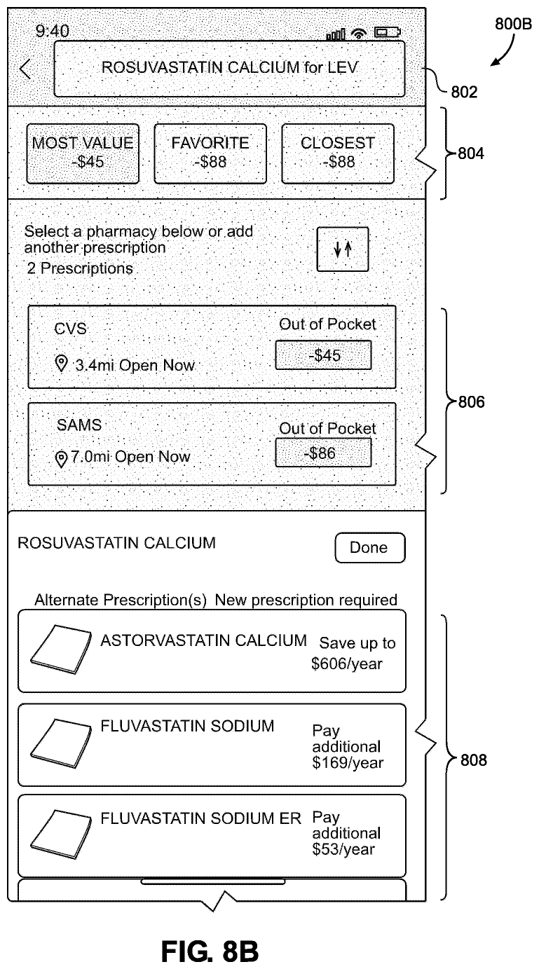

Turning now to FIGS. 8A and 8B, illustrated therein are examples of an alternate GUI that may be output on a mobile device 400 once a plurality of pharmacy fulfillment offers have been assembled for a consumer (e.g., the consumer who requested the offers by inputting information via the screen 400A and confirming the prescription details in screen 400B). FIG. 8A illustrates a version 800A of the interface before a user scrolls further down on the interface and FIG. 8B illustrates a version 800B of the interface after the user scrolls further down on the interface such that more of area 808 is visible to the user. The example interface 800A and 800B illustrates information and offers that may be output to a user for a given prescription indicated in area 802.

In accordance with one embodiment, the example interface of FIGS. 8A and 8B may be an alternative to an interface 400C (FIG. 4C) in that rather than showing, for each offer, the respective Reward amount and Cost to consumer amount as separate amounts for a given offer, the interface of FIGS. 8A and 8B combines the reward and cost into a single net amount that the consumer would pay out of pocket if he/she accepted the offer. For the example prescription illustrated in FIGS. 8A and 8B, these net amounts are indicated in area 804 and 806. For example, if a Reward for a given offer were calculated to be $5.00 and the Co-Pay, Co-insurance or other amount to be paid by the consumer for the prescription were to be calculated as $25.00, the net amount that that offer would cost the consumer would be shown as $20.00 in the interface of FIGS. 8A and 8B. In accordance with some embodiments, the net amount to the consumer may be an amount the consumer will receive, rather than an amount the consumer will pay (e.g., if the reward amount is greater than the reimbursement amount and/or there are monetary incentives included in the calculation of the net amount, such as if a healthcare service provider is offering a financial incentive to have consumers steered to his/her facility).

In accordance with some embodiments, the interface of FIGS. 8A and 8B also outputs, in area 808, options to the consumer for alternate or substitute prescriptions (i.e., different prescription drugs) and the corresponding additional savings or cost to the consumer (e.g., over a period of time such as a year). In accordance with some embodiments, a selection by the consumer of one of the alternate prescription drugs may require a new prescription from a treating physician before the prescription and offer may be fulfilled at a pharmacy. FIG. 8A illustrates in area 808 one of the alternate prescription offers and FIG. 8B illustrates in area 808 additional alternate prescription offers that may be visible to the consumer once he/she scrolls further down in the interface. It should be noted that in embodiments in which healthcare service providers are being identified and offered to the consumer, rather than pharmacy locations for fulfillment of a prescription, such alternate offers may comprise offers for alternate healthcare service providers, healthcare facilities or even different treatment options that the consumer should consider and discuss with his/her healthcare provider.

In accordance with some embodiments, three (3) pharmacy fulfillment offers have been assembled for the consumer and illustrated in the interfaces of FIGS. 8A and 8B, each for a different pharmacy and corresponding to a different out of pocket amount, as illustrated in area 804 of the interface. Each of the plurality of offers is also categorized differently and on factors other than distance. Thus, in the example of FIGS. 8A and 8B, one of the offers is for a pharmacy that offers the consumer the best “value” or price, another is for a pharmacy that is categorized as “favorite” (e.g., the one the consumer prefers or most often selects) and a third that is categorized as “closest” (e.g., shortest travel distance). In accordance with some embodiments, a consumer first selects the drug in the active configuration screen such as that illustrated in FIG. 8A and will subsequently be prompted to make additional selections (e.g., quantity and dosage selections). In some embodiments, upon making the selections, the consumer may be presented with the prices in the background and be provided with an opportunity to select his/her pharmacy of choice.

### Rules of Interpretation

Numerous embodiments have been described, and are presented for illustrative purposes only. The described embodiments are not intended to be limiting in any sense. The invention is widely applicable to numerous embodiments, as is readily apparent from the disclosure herein. These embodiments are described in sufficient detail to enable those skilled in the art to practice the invention, and it is to be understood that other embodiments may be utilized and that structural, logical, software, electrical and other changes may be made without departing from the scope of the present invention. Accordingly, those skilled in the art will recognize that the present invention may be practiced with various modifications and alterations. Although particular features of the present invention may be described with reference to one or more particular embodiments or figures that form a part of the present disclosure, and in which are shown, by way of illustration, specific embodiments of the invention, it should be understood that such features are not limited to usage in the one or more particular embodiments or figures with reference to which they are described. The present disclosure is thus neither a literal description of all embodiments of the invention nor a listing of features of the invention that must be present in all embodiments.

The terms “an embodiment”, “embodiment”, “embodiments”, “the embodiment”, “the embodiments”, “an embodiment”, “some embodiments”, “an example embodiment”, “at least one embodiment”, “one or more embodiments” and “one embodiment” mean “one or more (but not necessarily all) embodiments of the present invention(s)” unless expressly specified otherwise.

The terms “including”, “comprising” and variations thereof mean “including but not limited to”, unless expressly specified otherwise.

The term “consisting of” and variations thereof mean “including and limited to”, unless expressly specified otherwise.

The enumerated listing of items does not imply that any or all of the items are mutually exclusive. The enumerated listing of items does not imply that any or all of the items are collectively exhaustive of anything, unless expressly specified otherwise. The enumerated listing of items does not imply that the items are ordered in any manner according to the order in which they are enumerated.

The term “comprising at least one of” followed by a listing of items does not imply that a component or subcomponent from each item in the list is required. Rather, it means that one or more of the items listed may comprise the item specified. For example, if it is said “wherein A comprises at least one of: a, b and c” it is meant that (i) A may comprise a, (ii) A may comprise b, (iii) A may comprise c, (iv) A may comprise a and b, (v) A may comprise a and c, (vi) A may comprise b and c, or (vii) A may comprise a, b and c.

The terms “a”, “an” and “the” mean “one or more”, unless expressly specified otherwise.

The term “based on” means “based at least on”, unless expressly specified otherwise.

The methods described herein (regardless of whether they are referred to as methods, processes, algorithms, calculations, and the like) inherently include one or more steps. Therefore, all references to a “step” or “steps” of such a method have antecedent basis in the mere recitation of the term ‘method’ or a like term. Accordingly, any reference in a claim to a ‘step’ or ‘steps’ of a method is deemed to have sufficient antecedent basis.

Headings of sections provided in this document and the title are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more intermediaries.

A description of an embodiment with several components in communication with each other does not imply that all such components are required, or that each of the disclosed components must communicate with every other component. On the contrary a variety of optional components are described to illustrate the wide variety of possible embodiments of the present invention.

Further, although process steps, method steps, algorithms or the like may be described in a sequential order, such processes, methods and algorithms may be configured to work in alternate orders. In other words, any sequence or order of steps that may be described in this document does not, in and of itself, indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention, and does not imply that the illustrated process is preferred.

It will be readily apparent that the various methods and algorithms described herein may be implemented by, e.g., appropriately programmed general purpose computers and computing devices. Typically a processor (e.g., a microprocessor or controller device) will receive instructions from a memory or like storage device, and execute those instructions, thereby performing a process defined by those instructions. Further, programs that implement such methods and algorithms may be stored and transmitted using a variety of known media.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article.

The functionality and/or the features of a device may be alternatively embodied by one or more other devices which are not explicitly described as having such functionality/features. Thus, other embodiments of the present invention need not include the device itself.

The term “computer-readable medium” as used herein refers to any medium that participates in providing data (e.g., instructions) that may be read by a computer, a processor or a like device. Such a medium may take many forms, including but not limited to, non-volatile media, volatile media, and transmission media. Non-volatile media include, for example, optical or magnetic disks and other persistent memory. Volatile media may include dynamic random access memory (DRAM), which typically constitutes the main memory. Transmission media may include coaxial cables, copper wire and fiber optics, including the wires or other pathways that comprise a system bus coupled to the processor. Transmission media may include or convey acoustic waves, light waves and electromagnetic emissions, such as those generated during radio frequency (RF) and infrared (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying sequences of instructions to a processor. For example, sequences of instruction (i) may be delivered from RAM to a processor, (ii) may be carried over a wireless transmission medium, and/or (iii) may be formatted according to numerous formats, standards or protocols, such as Transmission Control Protocol, Internet Protocol (TCP/IP), Wi-Fi, Bluetooth, TDMA, CDMA, and 3G.

Where databases are described, it will be understood by one of ordinary skill in the art that (i) alternative database structures to those described may be readily employed, and (ii) other memory structures besides databases may be readily employed. Any schematic illustrations and accompanying descriptions of any sample databases presented herein are illustrative arrangements for stored representations of information. Any number of other arrangements may be employed besides those suggested by the tables shown. Similarly, any illustrated entries of the databases represent exemplary information only; those skilled in the art will understand that the number and content of the entries can be different from those illustrated herein. Further, despite any depiction of the databases as tables, other formats (including relational databases, object-based models and/or distributed databases) could be used to store and manipulate the data types described herein.

Likewise, object methods or behaviors of a database can be used to implement the processes of the present invention. In addition, the databases may, in a known manner, be stored locally or remotely from a device that accesses data in such a database.

For example, as an example alternative to a database structure for storing information, a hierarchical electronic file folder structure may be used. A program may then be used to access the appropriate information in an appropriate file folder in the hierarchy based on a file path named in the program.

It should also be understood that, to the extent that any term recited in the claims is referred to elsewhere in this document in a manner consistent with a single meaning, that is done for the sake of clarity only, and it is not intended that any such term be so restricted, by implication or otherwise, to that single meaning.

In a claim, a limitation of the claim which includes the phrase “means for” or the phrase “step for” means that 35 U.S.C. § 112, paragraph 6, applies to that limitation.

In a claim, a limitation of the claim which does not include the phrase “means for” or the phrase “step for” means that 35 U.S.C. § 112, paragraph 6 does not apply to that limitation, regardless of whether that limitation recites a function without recitation of structure, material or acts for performing that function. For example, in a claim, the mere use of the phrase “step of” or the phrase “steps of” in referring to one or more steps of the claim or of another claim does not mean that 35 U.S.C. § 112, paragraph 6, applies to that step(s).

With respect to a means or a step for performing a specified function in accordance with 35 U.S.C. § 112, paragraph 6, the corresponding structure, material or acts described in the specification, and equivalents thereof, may perform additional functions as well as the specified function.

Computers, processors, computing devices and like products are structures that can perform a wide variety of functions. Such products can be operable to perform a specified function by executing one or more programs, such as a program stored in a memory device of that product or in a memory device which that product accesses. Unless expressly specified otherwise, such a program need not be based on any particular algorithm, such as any particular algorithm that might be disclosed in the present application. It is well known to one of ordinary skill in the art that a specified function may be implemented via different algorithms, and any of a number of different algorithms would be a mere design choice for carrying out the specified function.

Therefore, with respect to a means or a step for performing a specified function in accordance with 35 U.S.C. § 112, paragraph 6, structure corresponding to a specified function includes any product programmed to perform the specified function. Such structure includes programmed products which perform the function, regardless of whether such product is programmed with (i) a disclosed algorithm for performing the function, (ii) an algorithm that is similar to a disclosed algorithm, or (iii) a different algorithm for performing the function.

While various embodiments have been described herein, it should be understood that the scope of the present invention is not limited to the particular embodiments explicitly described. Many other variations and embodiments would be understood by one of ordinary skill in the art upon reading the present description.

What is claimed is:

1. A system configured to facilitate obtainment of health care services by a user, the system comprising:
a processor;
a memory storing a program for directing the processor, the processor being operable with the program to:
receive first data defining a healthcare service needed by a user;
determine a user identifier that identifies the user corresponding to the first data;
retrieve, based on the user identifier, profile information that indicates at least one healthcare benefits insurance plan under which the user is covered;
determine a geographical location corresponding to the user;
identify a healthcare service provider that is acceptable under one or more terms of the healthcare benefits insurance plan;
access a plurality of reimbursement price lists, each reimbursement price list defining at least one healthcare service provider qualified to provide the healthcare service that is acceptable under one or more terms of the healthcare benefits insurance plan of the user;
select, based on the geographical location and the plurality of reimbursement price lists, a plurality of possible healthcare service providers;
generate a menu of offers, each offer defining a respective healthcare service provider at which the user can obtain the healthcare service, each offer corresponding to a respective consumer price that the user would pay for the healthcare service upon accepting the offer,
wherein each consumer price is based on a respective one of a reimbursement amount that the system would need to pay to the corresponding healthcare service provider upon the user obtaining the healthcare service, and
wherein at least one of the offers further defines a reward to be provided to the user upon the user obtaining the healthcare service at the healthcare service provider defined by the at least one offer;
output to the user, via a graphical user interface of a software application on a mobile device of the user, the menu of offers;
receive from the user, via an input mechanism of the graphical user interface, an acceptance of one of the offers, thereby identifying an accepted offer;
receive second data from the healthcare service provider defined by the accepted offer, the second data confirming that the user obtained the healthcare service from the healthcare service provider; and
provide the reward, if any, to the user based on the accepted offer.

2. The system of claim 1, wherein the first data comprises at least one diagnostic code corresponding to at least one diagnostic test to be administered to the user.

3. The system of claim 1, wherein the processor is operable with the program to determine the diagnostic code from a claim submitted by a healthcare provider associated with the user.

4. The system of claim 1, wherein the processor is further operable with the program to:
collect from the user a payment of the consumer price corresponding to the accepted offer.

5. The system of claim 1, wherein the processor is further operable with the program to:
update a record of a database to indicate that the second data has been received; and
provide a payment to the healthcare service provider from which the second data is received, the payment being based on the reimbursement amount based upon which the consumer price was based.

6. The system of claim 1, wherein the processor is further operable with the program to calculate the respective consumer price for each of the offers.

7. The system of claim 6, wherein each respective consumer price is calculated based on:
a predetermined baseline price that corresponds to the minimum of (i) the lowest price within a predetermined number of healthcare service provider locations that are closest to a location of the consumer, and (ii) the lowest price multiplied by a factor greater than 1 within a predetermined number of healthcare service provider locations that are the next closest to the location of the user, or (iii) a predetermined n^{th} nearest healthcare service providers locations to the location of the user.

8. The system of claim 7, wherein the reward that is associated with the at least one offer is associated with the offer that corresponds to a healthcare service provider location that has a respective reimbursement price lower than the baseline price.

9. The system of claim 7, wherein at least one of the consumer prices calculated includes a penalty and wherein the penalty is included in an offer of the plurality of offers that corresponds to the healthcare service location that has a respective reimbursement price higher than the baseline price

10. The system of claim 1, wherein the processor is further operable with the program to:
calculate a respective reward for at least one of the plurality of offers,
wherein a first value is calculated for a first reward included in a first offer and a second value is calculated for a second reward that is included in a second offer,
the first value being greater than the second value,
and wherein the first offer defines a first healthcare service location that has a greater savings relative to the baseline price and the second offer defines a second healthcare service location that is a lesser savings relative to the baseline price.

11. The system of claim 1, wherein the processor is operable with the program to calculate a respective consumer price for each of the offers such that a first price corresponding to a first offer is less than a second price corresponding to a second offer, and
wherein the first offer defines a first healthcare service location that has a greater savings relative to the baseline price and the second offer defines a second healthcare service location that has a lesser savings relative to the baseline price.

12. The system of claim 1, wherein the processor is further operable with the program to:
identify a financial incentive available from one of the possible healthcare service providers; and
include the financial incentive when calculating the consumer price for the corresponding offer.

13. The system of claim 12, wherein the financial incentive is directed to attracting consumers to the healthcare service provider within at least one of a preferred demographic profile and a preferred time frame and wherein the processor is further operable to confirm, prior to including the financial incentive when calculating the consumer price that the financial incentive qualifies for inclusion in the consumer price based on the at least one of the preferred demographic profile and the preferred time frame.

14. The system of claim 1, wherein the processor is further operable with the program to:
select and output to the consumer at least one suggestion for an alternate treatment option that the consumer should discuss with a healthcare provider associated with the first data.

15. The system of claim 14, wherein the alternate treatment option is selected from a database based on at least one diagnostic code comprising the first data.

16. The system of claim 14, wherein the processor is further operable with the program to:
receive from a healthcare provider of the user a verification that the alternate treatment option is acceptable; and
substitute, upon receiving the verification, the alternate treatment option for the first data and thereby generate the menu of offers in claim 1 for healthcare service providers corresponding to the alternate treatment option.

17. The system of claim 14, wherein the processor is operable with the program to combine, for each offer that defines the reward to be provided to the user, the consumer price and the reward for that offer, thereby calculating a net price to the user, and
wherein outputting the offer that defines the reward as part of the menu of offers comprising outputting an indication of the net price instead of the reward and the consumer price.
